(19) United States
(12) Patent Application Publication
(10) Pub. No.: US 2026/0325677 A1
(43) Pub. Date: Oct. 22, 2026

(54) GENE THERAPY METHODS FOR TREATING MITRAL VALVE DISEASE

(71) Applicant: Rejuvenate Bio Inc, San Diego, CA (US)

(72) Inventors: Noah Davidsohn, Brookline, MA (US); Dan Oliver, San Diego, CA (US)

(21) Appl. No.: 19/114,115

(22) PCT Filed: Sep. 21, 2023
(86) PCT No.: PCT/US2023/074731

§ 371 Date: Mar. 21, 2025

Related U.S. Application Data
(60) Provisional application No. 63/376,472, filed on Sep. 21, 2022.
(60) Provisional application No. 63/503,318, filed on May 19, 2023.

(51) Int. Cl. A61K 48/00; A61K 38/17; A61P 9/00; C07K 14/50; C12N 15/86

(52) U.S. Cl. CPC A61K 48/005; A61K 38/179; A61P 9/00; C07K 14/50; C12N 15/86; C12N 2750/14132; C12N 2750/14143

(57) ABSTRACT

Provided herein are methods of treating a mitral valve disease in a subject. The methods involve administering to the subject a gene therapy comprising a first nucleic acid encoding soluble transforming growth factor beta receptor 2 (sTGFbR2) and a second nucleic acid encoding fibroblast growth factor 21 (FGF21). Also provided are methods of treating a mitral valve disease in a subject comprising administering to the subject a gene therapy in combination with an additional therapeutic (e.g., pimobendan).

### RELATED APPLICATIONS

This application claims priority to U.S. Provisional Patent Application Ser. Nos. 63/376,472, filed Sep. 21, 2022, and 63/503,318, filed May 19, 2023, the entire disclosure of which is hereby incorporated herein by reference.

### REFERENCE TO SEQUENCE LISTING

This application contains a sequence listing which has been submitted electronically in ST.26 format and is hereby incorporated by reference in its entirety (said ST.26 copy, created on Sep. 19, 2023, is named “203422_SL.XML” and is 106,831 bytes in size).

### BACKGROUND

Mitral valve disease is a degenerative condition that results in dysfunction of the mitral valve over time. The mitral valve normally functions as a seal between the left atrium and the left ventricle, such that when the ventricle contracts, the mitral valve closes and prevents blood from leaking back into the atrium, a phenomenon known as regurgitation. If left untreated, as the left atrium becomes larger to accommodate the extra blood from regurgitation, increased pressure develops in the atrium, ultimately leading to congestive heart failure due to fluid buildup in the lungs.

In dogs, approximately 10% presented to primary care veterinary practices have heart disease, with myxomatous MVD (MMVD) being the most common heart disease of dogs in many parts of the world. MMVD accounts for approximately 75% of heart disease cases seen in dogs in North America. MMVD most commonly affects the mitral valve, although in at least 30% of cases, the tricuspid valve is also involved. The staging system for MMVD describes four basic stages of heart disease and heart failure: Stage A identifies dogs at high risk for developing heart disease but that currently have no identifiable structural disorder of the heart; Stage B identifies dogs with structural heart disease but that have never developed clinical signs caused by heart failure, with Stage B1 referring to asymptomatic dogs that have no to mild radiographic or echocardiographic evidence of cardiac remodeling in response to MMVD, and Stage B2 referring to asymptomatic dogs that have more advanced mitral valve regurgitation that is hemodynamically severe and long-standing enough to have caused radiographic and echocardiographic findings of left atrial and ventricular enlargement; Stage C identifies dogs with either current or past clinical signs caused by MMVD; and Stage D identifies dogs with end-stage MMVD, in which clinical sign of heart failure are refractory to standard treatment. Pimobendan is fully approved for the management of the signs of mild, moderate, or severe congestive heart failure in dogs due to MMVD, and was recently approved for treating dogs in the preclinical stage of MMVD who have a heart murmur and heart enlargement but are not yet in congestive heart failure. However, there is no cure for congestive heart failure due to MMVD.

Accordingly, there is a need in the art for improved methods for treating mitral valve disease.

### SUMMARY

The present disclosure provides methods of treating a mitral valve disease in a subject. The methods generally comprise administering to the subject a gene therapy comprising a first nucleic acid encoding soluble transforming growth factor beta receptor 2 (sTGFBR2) and a second nucleic acid encoding fibroblast growth factor 21 (FGF21). In certain embodiments, the gene therapy is administered in combination with an effective amount of pimobendan, or is administered to a subject that is currently undergoing treatment with pimobendan.

Accordingly, in one aspect, the instant disclosure provides a method of treating a mitral valve disease in a subject, comprising administering to the subject a gene therapy comprising a first nucleic acid encoding soluble transforming growth factor beta receptor 2 (sTGFBR2) and a second nucleic acid encoding fibroblast growth factor 21 (FGF21), wherein the subject has a left atrial to aortic root ratio (LA/Ao) of from about 1.6 to about 2.1. In some embodiments, the subject has a LA/Ao of about 1.6. In some embodiments, the subject has a LA/Ao of about 1.8. In some embodiments, the subject is mammal.

In certain embodiments, the subject is a canine. In some embodiments, the subject is a canine breed selected from the group consisting of: Cavalier King Charles Spaniel, Miniature Poodle, Shih Tzu, Maltese, Chihuahua, Cocker Spaniel, Miniature Schnauzer, Dachshund, Whippet, Pomeranian, and a com-bination thereof. In certain embodiments, the subject is a Cavalier King Charles Spaniel.

In certain embodiments, the mitral valve disease comprises one or more disease or condition selected from the group consisting of: myxomatous mitral valve disease, mitral valve stenosis, mitral valve prolapse, and mitral valve regurgitation.

In certain embodiments, the method further comprises determining the LA/Ao of the subject prior to administration of the gene therapy, and determining the LA/Ao of the subject at a duration after administration of the gene therapy. In certain embodiments, the LA/Ao of the subject after administration of the gene therapy is decreased as compared to the LA/Ao of the subject prior to administration of the gene therapy.

In certain embodiments, the subject has been administered an effective amount of pimobendan.

In certain embodiments, the method further comprises administering to the subject an effective amount of pimobendan.

In another aspect, the instant disclosure provides a method of treating a mitral valve disease in a subject, comprising administering to the subject a gene therapy comprising a first nucleic acid encoding soluble transforming growth fac-tor beta receptor 2 (sTGFBR2) and a second nucleic acid encoding fibroblast growth factor 21 (FGF21); and/or an effective amount of pimobendan.

In another aspect, the instant disclosure provides a method of treating a mitral valve disease in a subject that has received an effective amount of pimobendan, comprising administering to the subject a gene therapy comprising a first nucleic acid encoding soluble transforming growth factor beta receptor 2 (sTGFBR2) and a second nucleic acid encoding fibroblast growth factor 21 (FGF21).

In certain embodiments, the effective amount of pimobendan is 0.25 mg/kg. In some embodiments, the gene therapy and pimobendan are administered simultaneously. In certain embodiments, the pimobendan is administered orally. In certain embodiments, the pimobendan is administered twice a day. In certain embodiments, the effective amount of pimobendan is 0.25 mg/kg twice a day.

In another aspect, the instant disclosure provides a method of determining the likelihood of successful treatment of a subject having mitral valve disease with a gene therapy comprising a first nucleic acid encoding sTGFBR2 and a second nucleic acid encoding FGF21, comprising determining the LA/Ao of a subject, wherein an LA/Ao of greater than 2.1 indicates a decreased likelihood of successful treatment with the gene therapy and an LA/Ao of from about 1.6 to about 2.1 indicates an increased likelihood of successful treatment with the gene therapy. In certain embodiments, successful treatment of mitral valve disease comprises a decrease in LA/Ao of the subject as compared to the LA/Ao prior to gene therapy.

In another aspect, the instant disclosure provides a method of identifying a subject having mitral valve disease that is suitable for treatment with a gene therapy comprising a first nucleic acid encoding sTGFBR2 and a second nucleic acid encoding FGF21, comprising determining the LA/Ao of a subject, wherein if the subject has an LA/Ao of from about 1.6 to about 2.1, the subject is suitable for treatment with the gene therapy.

In certain embodiments, the subject is mammal. In certain embodiments, the subject is a canine. In certain embodiments, the canine is a breed selected from the group consisting of: Cavalier King Charles Spaniel, Miniature Poodle, Shih Tzu, Maltese, Chihuahua, Cocker Spaniel, Miniature Schnauzer, Dachshund, Whippet, Pomeranian, and a combination thereof. In some embodiments, the subject is a Cavalier King Charles Spaniel.

In certain embodiments, the mitral valve disease comprises one or more disease or condition selected from the group consisting of: myxomatous mitral valve dis-ease, mitral valve stenosis, mitral valve prolapse, and mitral valve regurgitation.

In certain embodiments, the gene therapy is administered intravenously.

In certain embodiments, the first nucleic acid comprises a first transcriptional regulatory element operably linked to the sTGFBR2 coding sequence. In some embodiments, the sTGFβR2 coding sequence comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 1, 2, 3, or 24. In certain embodiments, the sTGFβR2 coding sequence further comprises a heterologous or an innate secretion signal sequence, wherein the signal sequence is encoded by a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 4, 5, or 6. In certain embodiments, the sTGFβR2 coding sequence comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 9, 10, 11, 12, or 25. In certain embodiments, the sTGFβR2 coding sequence encodes an amino acid sequence having at least 85%, 86%, 87%, 88%, 89%, 90%, 91%, 92%, 93%, 94%, 95%, 96%, 97%, 98%, 99%, or 100% sequence identity to SEQ ID NO: 13, 14, 15, or 26. In certain embodiments, the sTGFβR2 coding sequence further encodes a secretion signal sequence having at least 85%, 86%, 87%, 88%, 89%, 90%, 91%, 92%, 93%, 94%, 95%, 96%, 97%, 98%, 99%, or 100% sequence identity to SEQ ID NO: 7 or 8. In certain embodiments, the sTGFβR2 coding sequence encodes an amino acid sequence having at least 85%, 86%, 87%, 88%, 89%, 90%, 91%, 92%, 93%, 94%, 95%, 96%, 97%, 98%, 99%, or 100% sequence identity to SEQ ID NO: 16, 17, 18, 19, or 27.

In certain embodiments, the second nucleic acid comprises a second transcriptional regulatory element operably linked to the FGF21 coding sequence. In certain embodiments, the FGF21 coding sequence comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 28, 29, 30, or 31. In certain embodiments, the FGF21 coding sequence encodes an amino acid sequence having at least 85%, 86%, 87%, 88%, 89%, 90%, 91%, 92%, 93%, 94%, 95%, 96%, 97%, 98%, 99%, or 100% sequence identity to SEQ ID NO: 32, 33, or 34.

In certain embodiments, the first and second transcriptional regulatory element each comprises one or more ApoE binding sites and/or an hAAT promoter. In certain embodiments, the first and second transcriptional regulatory element each comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 38, 39, 40, and/or 48.

In certain embodiments, the first and second nucleic acid each further comprises a post-transcriptional regulatory element. In certain embodiments, the post-transcriptional regulatory element comprises a polyadenylation signal and/or WPRE sequence. In certain embodiments, the polyadenylation signal is an SV40 polyadenylation signal. In certain embodiments, the WPRE sequence is a WPRE3 sequence. In certain embodiments, the post-transcriptional regulatory element comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 50, 51, or 70.

In certain embodiments, the first nucleic acid is comprised within a first vector, and the second nucleic acid is comprised within a second vector. In certain embodiments, the first vector and/or the second vector is each a viral vector, optionally wherein each is independently selected from the group consisting of adeno-associated virus (AAV), adenovirus, retrovirus, orthomyxovirus, paramyxovirus, papovavirus, picornavirus, lentivirus, herpes simplex virus, vaccinia virus, pox virus, and alphavirus. In certain embodiments, the first vector is an AAV vector comprised within a first recombinant AAV (rAAV), wherein the first rAAV comprises an AAV capsid comprising an AAV capsid protein; and a first rAAV genome; and/or the second vector is an AAV vector comprised within a second rAAV, wherein the second rAAV comprises an AAV capsid comprising an AAV capsid protein; and a second rAAV genome.

In certain embodiments, the gene therapy comprises: a first recombinant AAV (rAAV) comprising: an AAV capsid comprising an AAV capsid protein; and a first rAAV genome comprising a nucleic acid encoding the amino acid sequence set forth in SEQ ID NO: 26; and a second rAAV comprising: an AAV capsid comprising an AAV capsid protein; and a second rAAV genome comprising a nucleic acid encoding the amino acid sequence set forth in SEQ ID NO: 33.

In certain embodiments, the first rAAV genome comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 66.

In certain embodiments, the first rAAV genome further comprises a 5′ inverted terminal repeat (5′ ITR) nucleotide sequence, and a 3′ inverted terminal repeat (3′ ITR) nucleotide sequence. In certain embodiments, the 5′ ITR nucleotide sequence has at least 85%, 86%, 87%, 88%, 89%, 90%, 91%, 92%, 93%, 94%, 95%, 96%, 97%, 98%, 99%, or 100% sequence identity to SEQ ID NO: 58 or 59, and/or the 3′ ITR nucleotide sequence has at least 85%, 86%, 87%, 88%, 89%, 90%, 91%, 92%, 93%, 94%, 95%, 96%, 97%, 98%, 99%, or 100% sequence identity to SEQ ID NO: 60 or 61. In certain embodiments, the first rAAV genome comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 67.

In certain embodiments, the second rAAV genome comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 68.

In certain embodiments, the second rAAV genome further comprises a 5′ inverted terminal repeat (5′ ITR) nucleotide sequence, and a 3′ inverted terminal repeat (3′ ITR) nucleotide sequence. In certain embodiments, the 5′ ITR nucleotide sequence has at least 85%, 86%, 87%, 88%, 89%, 90%, 91%, 92%, 93%, 94%, 95%, 96%, 97%, 98%, 99%, or 100% sequence identity to SEQ ID NO: 58 or 59, and/or the 3′ ITR nucleotide sequence has at least 85%, 86%, 87%, 88%, 89%, 90%, 91%, 92%, 93%, 94%, 95%, 96%, 97%, 98%, 99%, or 100% sequence identity to SEQ ID NO: 60 or 61. In certain embodiments, the second rAAV genome comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 69.

In certain embodiments, the AAV capsid protein is derived from a clade A, clade B, clade C, clade D, clade E, clade F, clade G, clade H, clade I, AAVgo.1, AAV3, AAV4, AAV10,

AAV11, AAV12, rh.32, rh32.33, rh.33, rh.34, BAAV, or AAV5 capsid protein, or an engineered variant thereof. In certain embodiments, the AAV capsid protein comprises an amino acid sequence having at least 85%, 86%, 87%, 88%, 89%, 90%, 91%, 92%, 93%, 94%, 95%, 96%, 97%, 98%, 99%, or 100% sequence identity to SEQ ID NO: 63, 64, and/or 65.

In certain embodiments, the first and the second nucleic acid are comprised within a vector, optionally wherein the first and the second nucleic acid are separated by a polycistronic element. In certain embodiments, the vector comprises: the first nucleic acid comprising a nucleic acid encoding the amino acid sequence set forth in SEQ ID NO: 26; and the second nucleic acid comprising a nucleic acid encoding the amino acid sequence set forth in SEQ ID NO: 33. In certain embodiments, the first and the second nucleic acid are separated by a polycistronic element. In certain embodiments, the polycistronic element is an IRES or 2A sequence. In certain embodiments, the polycistronic element comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 55, 56, or 57.

In certain embodiments, the vector is an AAV vector comprised within a recombinant AAV (rAAV), wherein the rAAV comprises an AAV capsid comprising an AAV capsid protein; and a rAAV genome. In certain embodiments, the AAV capsid protein is derived from a clade A, clade B, clade C, clade D, clade E, clade F, clade G, clade H, clade I, AAVgo. 1, AAV3, AAV4, AAV10, AAV11, AAV12, rh.32, rh32.33, rh.33, rh.34, BAAV, or AAV5 capsid protein, or an engineered variant thereof. In certain embodiments, the AAV capsid protein comprises an amino acid sequence having at least 85%, 86%, 87%, 88%, 89%, 90%, 91%, 92%, 93%, 94%, 95%, 96%, 97%, 98%, 99%, or 100% sequence identity to SEQ ID NO: 63, 64, and/or 65.

### BRIEF DESCRIPTION OF THE DRAWINGS

FIGS. 1A and 1B are graphs showing the level of sTGFβR2 (FIG. 1A) and FGF21 (FIG. 1B) expression in subjects administered AAV8-sTGFβR2 and AAV8-FGF21 at the indicated doses, respectively.

FIG. 2 is a graph showing the LA/Ao over time in subjects administered AAV8-STGFβR2 and AAV8-FGF21. Each symbol represents an individual subject.

FIG. 3 is a graph showing the mean change in LA/Ao from baseline, over time in subjects administered pimobendan together with AAV8-sTGFβR2 and AAV8-FGF21 (Pimo+GT). Error bars represent interquartile range. Dashed line represents change in LA/Ao over time based on published data of subjects administered pimobendan alone.

FIG. 4 is a graph showing the mean change in fractional shortening percent (FS %) of the left ventricle from baseline, over time in subjects administered pimobendan together with AAV8-sTGFβR2 and AAV8-FGF21 (Pimo+GT). Error bars represent interquartile range. Dashed line represents change in fractional shortening over time based on published data of subjects administered pimobendan alone.

FIG. 5 is a graph showing the percentage of subjects administered pimobendan together with AAV8-sTGFβR2 and AAV8-FGF21 (Pimo+GT) that have not yet reached a primary endpoint over time. This data is overlaid on published data of subjects administered pimobendan alone, or placebo.

### DETAILED DESCRIPTION

The present disclosure provides methods of treating a mitral valve disease in a subject. The methods generally comprise administering to the subject a gene therapy comprising a first nucleic acid encoding soluble transforming growth factor beta receptor 2 (sTGFβR2) and a second nucleic acid encoding fibroblast growth factor 21 (FGF21). In certain embodiments, the gene therapy is administered in combination with an effective amount of pimobendan, or is administered to a subject that is currently undergoing treatment with pimobendan.

The present disclosure is based on the discovery that a certain population of subjects (e.g., canines) having mitral valve disease responded well to sTGFβR2 and FGF21 (sTGFβR2/FGF21) gene therapy. Subjects that had an initial left atrial-to-aortic root ratio (LA/Ao) of less than 2.1 that received sTGFβR2/FGF21 gene therapy resulted in a maintained LA/Ao or reduced LA/Ao over time. The present disclosure is also based on the discovery that pimobendan, the current best in class medication for treating MVD that has been shown to delay the onset of congestive heart failure through reduction in heart size, in combination with STGFβR2/FGF21 gene therapy results in synergistic reversal of left atrium enlargement in subjects (e.g., canines) with MVD.

### Definitions

As used herein, the term “replication-defective adeno-associated virus” refers to an AAV comprising a genome lacking Rep and Cap genes.

As used herein, the term “recombinant AAV genome” or “rAAV genome” refers to a coding sequence operably linked to an exogenous transcriptional regulatory element that mediates expression of the coding sequence when the rAAV genome is introduced into a cell. In certain embodiments, the rAAV genome does not integrate in the chromosomal DNA of the cell.

The skilled artisan will appreciate that the portion of a rAAV genome comprising the transcriptional regulatory element operably linked to a coding sequence can be in the sense or antisense orientation relative to direction of transcription of the coding sequence.

As used herein, the “percentage identity” between two nucleotide sequences or between two amino acid sequences is calculated by multiplying the number of matches between the pair of aligned sequences by 100, and dividing by the length of the aligned region, including internal gaps. Identity scoring only counts perfect matches, and does not consider the degree of similarity of amino acids to one another. Only internal gaps are included in the length, not gaps at the sequence ends.

As used herein, the term “coding sequence” refers to the portion of a complementary DNA (cDNA) that encodes a polypeptide, starting at the start codon and ending at the stop codon. A gene may have one or more coding sequences due to alternative splicing, alternative translation initiation, and variation within the population. A coding sequence may either be wild-type or codon optimized.

As used herein, the term “codon optimized” refers to alteration of a coding sequence of a gene (e.g., by nucleotide substitution) without changing the amino acid sequence of the polypeptide encoded by the coding sequence. Such codon alteration is advantageous in that it may increase the translation efficiency of a coding sequence, and/or prevent recombination with a corresponding sequence of an endogenous gene when a coding sequence is transduced into a cell.

As used herein, the term “transcriptional regulatory element” or “TRE” refers to a cis-acting nucleotide sequence, for example, a DNA sequence, that regulates (e.g., controls, increases, or reduces) transcription of an operably linked nucleotide sequence by an RNA polymerase to form an RNA molecule. A TRE relies on one or more trans-acting molecules, such as transcription factors, to regulate transcription. Thus, one TRE may regulate transcription in different ways when it is in contact with different trans-acting molecules, for example, when it is in different types of cells. A TRE may comprise one or more promoter elements and/or enhancer sequences. A skilled artisan would appreciate that the promoter and enhancer sequences in a gene may be close in location, and the term “promoter” may refer to a sequence comprising a promoter element and an enhancer sequence. Thus, the term “promoter” does not exclude an enhancer sequence in the sequence. The promoter and enhancer sequences do not need to be derived from the same gene or species, and the sequence of each promoter or enhancer sequence may be either identical or substantially identical to the corresponding endogenous sequence in the genome.

As used herein, the term “operably linked” is used to describe the connection between a TRE and a coding sequence to be transcribed. Typically, gene expression is placed under the control of a TRE comprising one or more promoter and/or enhancer sequences. The coding sequence is “operably linked” to the TRE if the transcription of the coding sequence is controlled or influenced by the TRE. The promoter and enhancer sequences of the TRE may be in any orientation and/or distance from the coding sequence, as long as the desired transcriptional activity is obtained. In certain embodiments, the TRE is upstream from the coding sequence.

As used herein, the term “polyadenylation signal” or “polyadenylation sequence” refers to a DNA sequence that when transcribed into RNA constitutes a polyadenylation signal sequence. The polyadenylation sequence can be native (e.g., with respect to the coding sequence of a gene) or exogenous. The exogenous polyadenylation sequence can be a mammalian or a viral polyadenylation sequence (e.g., an SV40 polyadenylation sequence).

As used herein, “exogenous polyadenylation sequence” refers to a polyadenylation sequence not identical or substantially identical to the endogenous polyadenylation sequence of a coding sequence of a gene. In certain embodiments, an exogenous polyadenylation sequence can be of the same species (e.g., human), or of a different species (e.g., a virus).

As used herein, the term “effective amount” in the context of the administration of a viral vector (e.g., recombinant AAV) to a subject refers to the amount of the viral vector that achieves a desired prophylactic or therapeutic effect. In the context of the administration of a compound, an effective amount refers to the amount of the compound that achieves a desired prophylactic or therapeutic effect.

As used herein, the term “polynucleotide,” in its broadest sense, includes any compound and/or substance that comprise a polymer of nucleotides linked via a phosphodiester bond.

As used herein, the term “treat,” “treating,” and “treatment” refer to therapeutic or preventative measures described herein. The methods of “treatment” employ administration of a polynucleotide to a subject having a disease or disorder, or predisposed to having such a disease or disorder, in order to prevent, cure, delay, reduce the severity of, or ameliorate one or more symptoms of the disease or disorder or recurring disease or disorder, or in order to prolong the survival of a subject beyond that expected in the absence of such treatment.

As used herein, the term “effective amount” in the context of the administration of a therapy to a subject refers to the amount of a therapy that achieves a desired prophylactic or therapeutic effect.

As used herein, the term “subject” includes any human or non-human animal. In certain embodiments, the subject is a non-human mammal. In certain embodiments, the subject is a canine. In certain embodiments, a canine subject is a canine breed such as Cavalier King Charles Spaniel, Miniature Poodle, Shih Tzu, Maltese, Chihuahua, Cocker Spaniel, Miniature Schnauzer, Dachshund, Whippet, Pomeranian, or a mixed breed thereof.

As used herein, the term “about” when used in connection with a numerical value is meant to encompass numerical values within a range having a lower limit that is 5% smaller than the indicated numerical value and having an upper limit that is 5% larger than the indicated numerical value.

### Polynucleotides, Vectors, and Compositions

In one aspect, the methods disclosed herein employ a first nucleic acid encoding soluble transforming growth factor beta receptor 2 (sTGFβR2) and a second nucleic acid encoding fibroblast growth factor 21 (FGF21).

In certain embodiments, the sTGFβR2 coding sequence encodes for a polypeptide comprising all or substantially all of the extracellular portion of TGFβR2 and does not encode for any transmembrane or intracellular aspects of TGFβR2. In certain embodiments, the sTGFβR2 coding sequence encodes for a polypeptide comprising the extracellular portion of wild type TGFβR2. In certain embodiments, the sTGFβR2 coding sequence encodes for a polypeptide comprising the extracellular portion of functional variant of TGFβR2. In certain embodiments, the sTGFβR2 coding sequence encodes a human, mouse, or canine TGFβR2.

In certain embodiments, the sTGFβR2 coding sequence encodes for a polypeptide comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 13, 14, or 15. In certain embodiments, the sTGFβR2 coding sequence comprises a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 1, 2, or 3.

In certain embodiments, the sTGFβR2 coding sequence encodes for a polypeptide comprising all or substantially all of the extracellular portion of TGFβR2, wherein the extracellular portion of TGFβR2 comprises a secretion signal sequence. In certain embodiments, the TGFβR2 secretion signal sequence is an innate secretion signal sequence. In certain embodiments, the TGFβR2 secretion signal sequence is a heterologous secretion signal sequence. For example, the heterologous secretion signal sequence can be obtained from a TGFβR2 secretion signal sequence of a different species. Exemplary TGFβR2 secretion signal sequences include, without limitation, the secretion signal sequences from human, mouse, and canine TGFβR2. As such, the sTGFβR2 coding sequence can further encode a heterologous or innate secretion signal sequence. In certain embodiments, the heterologous or innate secretion signal sequence comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 7 or 8. In certain embodiments, the heterologous or innate secretion signal sequence is encoded by a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 4, 5, or 6.

In certain embodiments, the sTGFβR2 coding sequence encodes for a polypeptide comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 16, 17, 18, or 19. In certain embodiments, the sTGFβR2 coding sequence comprises a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 9, 10, 11 or 12.

In certain embodiments, the sTGFβR2 coding sequence further encodes a peptide to achieve half-life extension. Such peptides include, without limitation, an IgG constant domain or fragment thereof (e.g., the Fc domain), human serum albumin (HSA), or albumin-binding polypeptides. In certain embodiments, the sTGFβR2 coding sequence further encodes an Fc domain (referred to herein as a sTGFβR2-Fc coding sequence). Exemplary Fc domains include wild type Fc domains from human, mouse, or canine IgG1, IgG2, IgG3 or IgG4. In certain embodiments, the Fc domain comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 22 or 23. In certain embodiments, the Fc domain is encoded by a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 20 or 21.

In certain embodiments, the sTGFβR2-Fc coding sequence encodes for a polypeptide comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 26 or 27. In certain embodiments, the sTGFβR2-Fc coding sequence comprises a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 24 or 25.

In certain embodiments, the FGF21 coding sequence encodes for a wild type FGF21, or a functional variant thereof. In certain embodiments, the FGF21 coding sequence encodes a human, mouse, or canine FGF21. In certain embodiments, the FGF21 coding sequence encodes for a polypeptide comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 32, 33, or 34. In certain embodiments, the FGF21 coding sequence comprises a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 28, 29, 30, or 31.

In certain embodiments, the first nucleic acid encoding soluble transforming growth factor beta receptor 2 (sTGFβR2) is each operably linked to a first transcriptional regulatory element (TRE). In certain embodiments, the second nucleic acid encoding fibroblast growth factor 21 (FGF21) is operably linked to a second TRE. In certain embodiments, the first TRE and the second TRE are the same. In certain embodiments, the first TRE and the second TRE are different. In certain embodiments, the first TRE and the second TRE comprises one or more common elements. The first TRE and the second TRE can be active in any mammalian cells (e.g., human cells, canine cells).

In certain embodiments, the TRE is active in a broad range of mammalian cells. Such TREs may comprise a constitutive promoter and/or enhancer sequences including a cytomegalovirus (CMV) promoter (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 41); a CMV enhancer sequence, a CBA promoter, and the splice acceptor from exon 3 of the rabbit beta-globin gene, collectively called a CAG promoter (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 42); a human calmodulin 1 (CALM1) promoter (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 43); a chicken beta actin (CBA) promoter (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 44); a CASI promoter (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 45); an smCBA promoter (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 46); a human elongation factor 1 alpha (EFla) promoter (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 35, 36, or 37); an SV40 promoter; a human phosphoglycerate kinase (PGK1) promoter; a human ubiquitin C (Ubc) promoter; a human beta actin promoter; a human neuron-specific enolase (ENO2) promoter; a human beta-glucuronidase (GUSB) promoter; and/or a human Methyl-CpG Binding Protein 2 (MeCP2) promoter. Any of these TREs or elements within these TREs can be combined in any order to drive efficient transcription.

Alternatively, the TRE may be a tissue-specific TRE, i.e., it is active in specific tissue(s) and/or organ(s). A tissue-specific TRE comprises one or more tissue-specific promoter and/or enhancer sequences, and optionally one or more constitutive promoter and/or enhancer sequences. A skilled artisan would appreciate that tissue-specific promoter and/or enhancer sequences can be isolated from genes specifically expressed in the tissue by methods well known in the art.

In certain embodiments, the TRE is liver-specific, i.e., it is active in cells of the liver. Liver-specific TREs include, without limitation, those provided on the Liver Specific Gene Promoter Database (LSPD, rulai.cshl.edu/LSPD/); a human alpha-1-antitrypsin (hAAT) promoter (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 38); an apolipoprotein E (ApoE) binding site (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 39 or 40); a human albumin (hAlb) or minimal promoter; a transthyretin (TTR) promoter or TTR-minimal promoter (TTRm); an apolipoprotein A1 (APOAI) promoter or minimal promoter; a complement factor B (CFB) promoter; a ketohexokinase (KHK) promoter; a hemopexin (HPX) promoter or minimal promoter; a nicotinamide N-methyltransferase (NNMT) promoter or minimal promoter; a (liver) carboxylesterase 1 (CES1) promoter or minimal promoter; a protein C (PROC) promoter or minimal promoter; an apolipoprotein C3 (APOC3) promoter or minimal promoter; a mannan-binding lectin serine protease 2 (MASP2) promoter or minimal promoter; a hepcidin antimicrobial peptide (HAMP) promoter or minimal promoter; and a serpin peptidase inhibitor, clade C (antithrombin), member 1 (SERPINC1) promoter or minimal promoter.

The TRE may be an inducible promoter. The use of an inducible promoter provides a molecular switch capable of turning on expression of the polynucleotide sequence which it is operatively linked when such expression is desired, or turning off the expression when expression is not desired. Examples of inducible promoters include, without limitation, a metallothionine promoter, a glucocorticoid promoter, a progesterone promoter, and a tetracycline promoter.

In certain embodiments, the first nucleic acid and/or the second nucleic acid comprises two or more TREs, optionally comprising at least one of the TREs disclosed herein. A skilled person in the art would appreciate that any of these TREs can be combined in any order, and combinations of a constitutive TRE and a tissue-specific TRE can drive efficient and tissue-specific transcription.

In certain embodiments, the TRE can further comprise an intron sequence. Such introns can increase transgene expression, for example, by reducing transcriptional silencing and enhancing mRNA export from the nucleus to the cytoplasm. The intron can comprise a native intron sequence of sTGFβR2 or FGF21, an intron sequence from the same genes of a different species, an intron sequence from a different gene from the same species, and/or a synthetic intron sequence. A skilled worker will appreciate that synthetic intron sequences can be designed to mediate RNA splicing by introducing any consensus splicing motifs known in the art (e.g., in Sibley et al. Nature Reviews Genetics. 2016, 17:407-21, which is incorporated by reference herein in its entirety). Exemplary intron sequences are provided in Lu et al., Molecular Therapy. 2013, 21 (5): 954-63, and Lu et al., Hum. Gene Ther. 2017, 28 (1): 125-34, which are incorporated by reference herein in their entirety. Suitable intron sequence include, without limitation, a minute virus of mouse (MVM) intron (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 47); a β-globin intron sequence (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 48); and an SV40 intron sequence.

In certain embodiments, the first nucleic acid and/or the second nucleic acid further comprises a post-transcriptional regulatory element. The post-transcriptional regulatory element may be any sequence that effectively terminates transcription, and a skilled artisan would appreciate that such sequences can be isolated from any genes that are expressed in the cell in which transcription of the coding sequence is desired.

In certain embodiments, the post-transcriptional regulatory element comprises a polyadenylation signal sequence. In certain embodiments, the polyadenylation signal sequence is identical or substantially identical to the endogenous polyadenylation sequence of the sTGFβR2 or FGF21 gene. In certain embodiments, the polyadenylation signal sequence is an exogenous polyadenylation signal sequence. In certain embodiments, the polyadenylation signal sequence is an SV40 polyadenylation sequence (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 51); a bovine growth hormone polyadenylation sequence (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 52); a rabbit beta globin polyadenylation sequence (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 53); or a human growth hormone polyadenylation sequence (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 54).

In certain embodiments, the post-transcriptional regulatory element comprises a Woodchuck Hepatitis Virus (WHV) post-transcriptional regulatory element (WPRE). In certain embodiments, the post-transcriptional regulatory element comprises a WPRE sequence (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 49); or a WPRE3 sequence (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 50 or 70).

The first nucleic acid and/or the second nucleic acid described herein can be transcribed from an expression vector (e.g., a recombinant expression vector). In certain embodiments, the first nucleic acid is comprised within a first vector, and the second nucleic acid is comprised within a second vector. In certain embodiments, the first nucleic acid and the second nucleic acid are comprised within a single vector. Where the first nucleic acid and the second nucleic acid are comprised within a single vector, the first nucleic acid and the second nucleic acid may be separated by a polycistronic element.

In certain embodiments, the polycistronic element comprises a nucleotide sequence that encodes for an internal ribosome entry site (IRES). An IRES is an element that promotes direct internal ribosome entry to the initiation codon, such as ATG, of a protein coding region, thereby leading to cap-independent translation of the gene. Various internal ribosome entry sites are known to those of skill in the art, including, without limitation, IRES obtainable from viral or cellular mRNA sources, e.g., immunoglobulin heavy-chain binding protein (BiP); vascular endothelial growth factor (VEGF); fibroblast growth factor 2; insulin-like growth factor; translational initiation factor eIF4G; yeast transcription factors TFIID and HAP4; and IRES obtainable from, e.g., cardiovirus, rhinovirus, aphthovirus, HCV, Friend murine leukemia virus (FrMLV), and Moloney murine leukemia virus (MoMLV). In certain embodiments, the polycistronic element comprises a nucleotide sequence that encodes for a 2A sequence. A 2A sequence refers to an oligopeptide that allow multiple proteins to be encoded as polyproteins, which dissociate into component proteins upon translation. Various 2A sequences are known to those of skill in the art, including, without limitation, those found in members of the Picornaviridae virus family, e.g., foot-and-mouth disease virus (FMDV), equine rhinitis A virus (ERAVO),

Thosea asigna virus (TaV), and porcine tescho virus-1 (PTV-1); and carioviruses such as Theilovirus and encephalomyocarditis viruses. 2A sequences derived from FMDV, ERAV, PTV-1, and TaV are referred to herein as “F2A,” “E2A,” “P2A,” and “T2A,” respectively. In certain embodiments, the polycistronic element comprises a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 55, 56, or 57.

In certain embodiments, the vector is a non-viral vector. Exemplary non-viral vectors include, but are not limited to, plasmid DNA, transposons, episomal plasmids, minicircles, ministrings, and oligonucleotides (e.g., mRNA, naked DNA). In certain embodiments, the non-viral vector is a DNA plasmid vector. In certain embodiments, the non-viral vector is a transposon-based vector. In certain embodiments, the non-viral vector is a PiggyBac-based vector, or a Sleeping Beauty-based vector.

In certain embodiments, the vector is a viral vector. Viral vectors can be replication competent or replication incompetent. Viral vectors can be integrating or non-integrating. A number of viral based systems have been developed for gene transfer into mammalian cells, and a suitable viral vector can be selected by a person of ordinary skill in the art. Exemplary viral vectors include, but are not limited to, adenovirus vectors (e.g., adenovirus 5), adeno-associated virus (AAV) vectors (e.g., AAV1, AAV2, AAV3, AAV4, AAV5, AAV6, AAV7, AAV8, AAV9), retrovirus vectors (e.g., MMSV, MSCV), lentivirus vectors (e.g., HIV-1, HIV-2), gammaretrovirus vectors, herpes virus vectors (e.g., HSV1, HSV2), alphavirus vectors (e.g., SFV, SIN, VEE, MI), flavivirus (e.g., Kunjin, West Nile, Dengue virus), rhabdovirus vectors (e.g., rabies virus, VSV), measles virus vector, Newcastle disease virus vectors, poxvirus vectors, and picornavirus vectors (e.g., Coxsackievirus). In certain embodiments, the viral vector is selected from the group consisting of adeno-associated virus (AAV), adenovirus, retrovirus, orthomyxovirus, paramyxovirus, papovavirus, picornavirus, lentivirus, herpes simplex virus, vaccinia virus, pox virus, and alphavirus.

In certain embodiments, the vector is an AAV vector. In certain embodiments, the vector is a single-stranded AAV. In certain embodiments, the vector is a self-complementary AAV.

In certain embodiments, the vector is an AAV vector comprised within a recombinant AAV (rAAV). In certain embodiments, the rAAV comprises an AAV capsid comprising an AAV capsid protein, and a rAAV genome.

A capsid protein from any capsid known the art can be used in the rAAV compositions disclosed herein, including, without limitation, a capsid protein from an AAV1,

AAV2, AAV3, AAV4, AAV5, AAV6, AAV7, AAV8, or AAV9 serotype. The capsid protein can be from a clade A, clade B, clade C, clade D, clade E, clade F, clade G, clade H, clade I, AAVgo.1, AAV3, AAV4, AAV10, AAV11, AAV12, rh.32, rh32.33, rh.33, rh.34, BAAV, or AAV5 capsid protein, or an engineered variant thereof. In certain embodiments, the capsid protein is from AAV8. In certain embodiments, the capsid protein is encoded by a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 62. In certain embodiments, the capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of amino acids 1-738 of SEQ ID NO: 63; an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of amino acids 138-738 of SEQ ID NO: 63; and/or an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of amino acids 204-738 of SEQ ID NO: 63. In certain embodiments, the capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 63; an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 64; and/or an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 65.

In certain embodiments, the rAAV comprises an AAV capsid comprising an AAV capsid protein, and a rAAV genome comprising a nucleic acid encoding sTGFβR2. In certain embodiments, the rAAV comprises an AAV capsid comprising an AAV capsid protein, and a rAAV genome comprising a nucleic acid encoding sTGFβR2-Fc. In certain embodiments, the rAAV comprises an AAV capsid comprising an AAV capsid protein, and a rAAV genome comprising a nucleic acid encoding an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 26. In certain embodiments, the rAAV comprises an AAV capsid comprising an AAV capsid protein, and a rAAV genome comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 66.

In certain embodiments, the rAAV comprises an AAV capsid comprising an AAV capsid protein, and a rAAV genome comprising a nucleic acid encoding FGF21. In certain embodiments, the rAAV comprises an AAV capsid comprising an AAV capsid protein, and a rAAV genome comprising a nucleic acid encoding an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 33. In certain embodiments, the rAAV comprises an AAV capsid comprising an AAV capsid protein, and a rAAV genome comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 67.

In certain embodiments, the rAAV genome further comprises a 5′ inverted terminal repeat (5′ITR) nucleotide sequence, and a 3′ inverted terminal repeat (3′ ITR) nucleotide sequence 3′ of the coding sequence. In certain embodiments, the rAAV genome comprises a 5′ ITR 5′ of the TRE, and a 3′ ITR 3′ of the coding sequence. ITR sequences from any AAV serotype or variant thereof can be used in the rAAV genomes disclosed herein. The 5′ and 3′ ITR can be from an AAV of the same serotype or from AAVs of different serotypes. Exemplary ITRs for use in the rAAV genomes disclosed herein are set forth in SEQ ID NO: 58, 59, 60, and 61.

In certain embodiments, the 5′ ITR or 3′ ITR is from AAV2. In certain embodiments, both the 5′ ITR and the 3′ ITR are from AAV2. In certain embodiments, the 5′ITR nucleotide sequence has at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 58 or 59. In certain embodiments, the 3′ ITR nucleotide sequence has at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 60 or 61. In certain embodiments, the 5′ ITR nucleotide sequence has at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 59, and the 3′ ITR nucleotide sequence has at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 61.

In certain embodiments, the rAAV genome comprises from 5′ to 3′: a 5′ ITR (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 59); a transcriptional regulatory element operably linked to a nucleic acid encoding sTGFβR2 or sTGFβR2-Fc (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 2, 11, 20, 24, 25, 38, 39, 40, 47, or 48); a post-transcriptional regulatory element (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 50, 51, or 70); and a 3′ ITR (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 61). In certain embodiments, the rAAV genome comprises a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 67.

In certain embodiments, the rAAV genome comprises from 5′ to 3′: a 5′ ITR (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 59); a transcriptional regulatory element operably linked to a nucleic acid encoding FGF21 (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 30, 38, 39, 40, 47, or 48); a post-transcriptional regulatory element (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 50, 51, or 70); and a 3′ ITR (e.g., comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 61). In certain embodiments, the rAAV genome comprises a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 69.

In certain embodiments, the rAAV comprises: (a) an AAV capsid protein comprising the amino acid sequence of amino acids 1-738 of SEQ ID NO: 63, the amino acid sequence of amino acids 138-738 of SEQ ID NO: 63, and/or the amino acid sequence of amino acids 204-738 of SEQ ID NO: 63; and (b) an rAAV genome comprising the nucleotide sequence set forth in any one of SEQ ID NOs: 66, 67, 68, or 69. In certain embodiments, the rAAV comprises (a) an AAV capsid protein comprising the amino acid sequence of amino acids 1-738 of SEQ ID NO: 63, and an rAAV genome comprising the nucleotide sequence set forth in any one of SEQ ID NOs: 66, 67, 68, or 69; (b) an AAV capsid protein comprising the amino acid sequence of amino acids 138-738 of SEQ ID NO: 63, and an rAAV genome comprising the nucleotide sequence set forth in any one of SEQ ID NOs: 66, 67, 68, or 69; and/or (c) an AAV capsid protein comprising the amino acid sequence of amino acids 204-738 of SEQ ID NO: 63, and an rAAV genome comprising the nucleotide sequence set forth in any one of SEQ ID NOs: 66, 67, 68, or 69;

In another aspect, the present disclosure provides a polynucleotide comprising a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 66, 67, 68, or 69.

The polynucleotide can comprise DNA, RNA, modified DNA, modified RNA, or a combination thereof. In certain embodiments, the polynucleotide is an expression vector. In certain embodiments, the polynucleotide is comprised within a viral vector. In certain embodiments, the polynucleotide is comprised within a plasmid vector.

In another aspect, the present disclosure provides pharmaceutical compositions comprising a rAAV as disclosed herein together with a pharmaceutically acceptable excipient, adjuvant, diluent, vehicle or carrier, or a combination thereof. A “pharmaceutically acceptable carrier” includes any material which, when combined with an active ingredient of a composition, allows the ingredient to retain biological activity and without causing disruptive physiological reactions, such as an unintended immune reaction. Pharmaceutically acceptable carriers include water, phosphate buffered saline, emulsions such as oil/water emulsion, and wetting agents. Compositions comprising such carriers are formulated by well-known conventional methods such as those set forth in Remington's Pharmaceutical Sciences, current Ed., Mack Publishing Co., Easton Pa. 18042, USA; A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., 7th ed., Lippincott, Williams, & Wilkins; and Handbook of Pharmaceutical Excipients (2000) A. H. Kibbe et al., 3rd ed. Amer. Pharmaceutical Assoc.

### Methods of Treatment

In another aspect, the present disclosure provides methods for treating a mitral valve disease in a subject. The methods generally comprise administering to the subject an effective amount of a gene therapy comprising a first nucleic acid encoding soluble transforming growth factor beta receptor 2 (sTGFβR2) and a second nucleic acid encoding fibroblast growth factor 21 (FGF21).

In certain embodiments, a method for treating a MVD in a subject comprises administering: (a) a first rAAV comprising an AAV capsid comprising an AAV capsid protein, and a first rAAV genome (e.g., comprising a nucleic acid encoding sTGFβR2); and (b) a second rAAV comprising an AAV capsid comprising an AAV capsid protein, and a second rAAV genome (e.g., comprising a nucleic acid encoding FGF21). In certain embodiments, a method for treating a MVD in a subject comprises administering: (a) a first rAAV comprising an AAV capsid comprising an AAV capsid protein, and a first rAAV genome comprising a nucleic acid encoding the amino acid sequence set forth in SEQ ID NO: 26; and (b) a second rAAV comprising an AAV capsid comprising an AAV capsid protein, and a second rAAV genome comprising a nucleic acid encoding the amino acid sequence set forth in SEQ ID NO: 33. In certain embodiments, a method for treating a MVD in a subject comprises administering: (a) a first rAAV comprising an AAV capsid comprising an AAV capsid protein, and a first rAAV genome comprising the nucleotide sequence set forth in SEQ ID NO: 66 or 68; and (b) a second rAAV comprising an AAV capsid comprising an AAV capsid protein, and a second rAAV genome comprising the nucleotide sequence set forth in SEQ ID NO: 67 or 69.

In certain embodiments, a method for treating a MVD in a subject comprises administering an rAAV comprising an AAV capsid comprising an AAV capsid protein, and an rAAV genome comprising: a first nucleic acid encoding sTGFβR2 and a second nucleic acid encoding FGF21, wherein the first nucleic acid and the second nucleic acid are separated by a polycistronic element. In certain embodiments, a method for treating a MVD in a subject comprises administering an rAAV comprising an AAV capsid comprising an AAV capsid protein, and an rAAV genome comprising: a first nucleic acid encoding the amino acid sequence set forth in SEQ ID NO: 26 and a second nucleic acid encoding the amino acid sequence set forth in SEQ ID NO: 33, wherein the first nucleic acid and the second nucleic acid are separated by a polycistronic element.

In certain embodiments, a method for treating a MVD in a subject further comprises determining the LA/Ao of the subject prior to administration of the gene therapy, and determining the LA/Ao of the subject at a duration (e.g., 1 week, 2 weeks, 3 weeks, 4 weeks, 1 month, 2 months, 3 months, 4 months, 5 months, 6 months, 1 year, 2 years, 3 years, 4 years) after administration of the gene therapy. Methods of determining the LA/Ao of a subject are known in the art. For example, the LA/Ao can be measured from a right parasternal short-axis view at the heart base. Briefly, the internal short-axis diameter of the aorta along the commissure between the noncoronary and right coronary aortic valve cusps on the 1st frame after aortic valve closure can be measured. Then, the internal short-axis diameter of the LA in the same frame in a line extending from and parallel to the commissure between the noncoronary and left coronary aortic valve cusps to the distant margin of the left atrium is measured.

Various methods for diagnosing MVD aside from LA/Ao measurement are known in the art, and include, without limitation, via echocardiograms, electrocardiograms, chest x-rays, cardiac magnetic resonance imaging, exercise tests, stress tests, and/or cardiac catheterization. The staging system for MVD generally refers to four basic groups: Stage A-at risk: risk factors for MVD are present; Stage B—progressive: MVD is mild or moderate, and there are no heart valve symptoms; Stage C—asymptomatic severe: MVD is severe, and there are no heart valve symptoms; and Stage D—symptomatic severe: MVD is severe and is causing symptoms. Stage B can be further subdivided into Stage B1 and Stage B2; Stage B1 is diagnosed when a heart murmur is detected but there is no radiographic or echocardiographic evidence of cardiac remodeling or remodeling that is not severe enough to meet current clinical trial criteria for treatment; Stage B2 is diagnosed when a heart murmur is detected and there is radiographic or echocardiographic evidence of cardiac remodeling that is severe enough to meet current clinical trial criteria for treatment. It is known in the art that an LA/Ao of greater than 1.6 is indicative of MVD stage B2.

In certain embodiments, a method for treating a MVD in a subject comprises administering to the subject the gene therapy, wherein the subject has a LA/Ao of from about 1.6 to about 2.1, e.g., about 1.6, about 1.7, about 1.8, about 1.9, about 2, about 2.1. In certain embodiments, the subject has a LA/Ao of about 1.6. In certain embodiments, the subject has a LA/Ao of about 1.8. In certain embodiments, the subject has a LA/Ao of about 2.1 In certain embodiments, the subject has a LA/Ao of less than about 2.1. It was found that regardless of dosage, subjects with an initial LA/Ao of less than 2.1 responded to the gene therapy over time, showing a reversal of left atrium enlargement over time. It will readily be understood by those of skill in the art that LA/Ao measurement is only one method of diagnosing MVD. As such, those of skill in the art will be able to determine, when using other methods of diagnosing MVD, what measurement result is equivalent to a subject having an initial LA/Ao of less than 2.1. The present disclosure also contemplates methods for treating a MVD in those subjects having a diagnostic measurement that results in an equivalent to a subject having an initial LA/Ao of less than 2.1 (e.g., a diagnostic equivalent to an initial LA/Ao of less than 2.1).

In certain embodiments, a method for treating a MVD in a subject further comprises administering to the subject an effective amount of one or more additional therapeutics to treat MVD. Such additional therapeutics to treat MVD may include, without limitation, diuretics, blood thinners (i.e., anticoagulants), and blood pressure medications. For example, in dogs, pimobendan is used in the management of heart failure due to MMVD. Pimobendan is often used in conjunction with an ACE inhibitor such as enalapril or benazepril. In certain embodiments, a method for treating MVD in a subject further comprises administering to the subject an effective amount of pimobendan. Other additional therapeutics for treating MVD include furosemide, spironolactone (i.e., an aldosterone antagonist), and an angiotensin-converting enzyme (ACE) inhibitor.

In certain embodiments, a method for treating a MVD in a subject comprises administering to the subject: (a) an effective amount of a gene therapy comprising a first nucleic acid encoding soluble transforming growth factor beta receptor 2 (sTGFβR2) and a second nucleic acid encoding fibroblast growth factor 21 (FGF21); and (b) an effective amount of pimobendan. In certain embodiments, a method for treating a MVD in a subject comprises administering to the subject: (a) a first rAAV comprising an AAV capsid comprising an AAV capsid protein and a first rAAV genome comprising a nucleic acid encoding sTGFβR2, and a second rAAV comprising an AAV capsid comprising an AAV capsid protein and a second rAAV genome comprising a nucleic acid encoding FGF21; and (b) an effective amount of pimobendan. In certain embodiments, a method for treating a MVD in a subject comprises administering to the subject: (a) an rAAV comprising an AAV capsid comprising an AAV capsid protein, and an rAAV genome comprising: a first nucleic acid encoding sTGFβR2 and a second nucleic acid encoding FGF21, wherein the first nucleic acid and the second nucleic acid are separated by a polycistronic element; and (b) an effective amount of pimobendan.

In dogs, pimobendan is may be administered orally at a total daily dose of 0.23 mg/lb (0.5 mg/kg) body weight. In humans, pimobendan is may be administered at a dose of 2.5 mg/day. The total daily dose is typically divided into two portions that are administered approximately 12 hours apart. In certain embodiments, the effective amount of pimobendan is between about 0.05 and about 0.5 mg/kg. In some embodiments, the effective amount of pimobendan is between about 0.05 and about 0.5 mg/kg, administered twice daily for a total daily dose of between about 0.10 and about 1.0 mg/kg. In some embodiments, the effective amount of pimobendan is about 0.25 mg/kg, administered twice daily for a total daily dose of about 0.5 mg/kg. In some embodiments, the effective amount of pimobendan is 0.25 mg/kg, administered twice daily for a total daily dose of 0.5 mg/kg.

In certain embodiments, the effective amount of pimobendan is between about 0.2 mg/kg to about 0.6 mg/kg bodyweight once a day. In certain embodiments, the effective amount of pimobendan is between about 0.2 mg/kg to about 0.6 mg/kg bodyweight administered per day. In certain embodiments, the effective amount of pimobendan is between about 0.2 mg/kg to about 0.5 mg/kg bodyweight administered per day. In certain embodiments, the daily pimobendan dose is administered as two doses of between about 0.1 mg/kg to about 0.3 mg/kg bodyweight. In certain embodiments, the daily pimobendan dose is administered as two doses of between about 0.1 mg/kg to about 0.3 mg/kg bodyweight every 12 hours. In certain embodiments, the daily pimobendan dose is administered as two doses of 0.25 mg/kg bodyweight every 12 hours.

In certain embodiments, the additional therapeutic to treat MVD (e.g., pimobendan) is administered at the same time as the gene therapy. In certain embodiments, the additional therapeutic to treat MVD (e.g., pimobendan) is administered at a different time as the gene therapy.

In certain embodiments, a method of treating a MVD in a subject comprises administering to a subject that has received an effective amount of a non-gene therapy therapeutic to treat MVD, an effective amount of a gene therapy comprising a first nucleic acid encoding soluble transforming growth factor beta receptor 2 (sTGFβR2) and a second nucleic acid encoding fibroblast growth factor 21 (FGF21). In certain embodiments, a method of treating a MVD in a subject comprises administering to a subject that has received an effective amount of pimobendan, an effective amount of a gene therapy comprising a first nucleic acid encoding soluble transforming growth factor beta receptor 2 (sTGFβR2) and a second nucleic acid encoding fibroblast growth factor 21 (FGF21).

In another aspect, the present disclosure provides a method of determining the likelihood of successful treatment of a subject having mitral valve disease with a gene therapy comprising a first nucleic acid encoding sTGFβR2 and a second nucleic acid encoding FGF21, comprising determining the LA/Ao of a subject, wherein an LA/Ao of greater than 2.1 indicates a decreased likelihood of successful treatment with the gene therapy and an LA/Ao of from about 1.6 to about 2.1 indicates an increased likelihood of successful treatment with the gene therapy. In certain embodiments, successful treatment of a MVD comprises a decrease in LA/Ao of the subject as compared to the LA/Ao measured prior to the gene therapy. In certain embodiments, successful treatment of MVD comprises a slower increase of LA/Ao of the subject over time, as compared to the level of increase of LA/Ao of a subject who has not received gene therapy. In certain embodiments, successful treatment of MVD comprises maintaining the LA/Ao of the subject over time, as compared to the change in LA/Ao of a subject who has not received gene therapy.

In another aspect, the present disclosure provides a method of identifying a subject having mitral valve disease that is suitable for treatment with a gene therapy comprising a first nucleic acid encoding sTGFβR2 and a second nucleic acid encoding FGF21, comprising determining the LA/Ao of a subject, wherein if the subject has an LA/Ao of from about 1.6 to about 2.1, the subject is suitable for treatment with the gene therapy.

In certain embodiments, the mitral valve disease comprises one or more disease or condition selected from the group consisting of: myxomatous mitral valve disease, mitral valve stenosis, mitral valve prolapse, and mitral valve regurgitation.

In certain embodiments, the gene therapy is administered to the subject intravenously, intraperitoneally, subcutaneously, intramuscularly, intrathecally, or intradermally.

In certain embodiments, the subject is a member of any mammalian or non-mammalian species. Suitable subjects include, without limitation, humans, non-human primates, canines, felines, ungulates (e.g., equine, bovine, swine (e.g., pig)), avians, rodents (e.g., rats, mice), and other subjects. In certain embodiments, the subject is human. In certain embodiments, the subject is canine. In certain embodiments, the subject is a canine breed selected from the group consisting of: Cavalier King Charles Spaniel, Miniature Poodle, Shih Tzu, Maltese, Chihuahua, Cocker Spaniel, Miniature Schnauzer, Dachshund, Whippet, Pomeranian, and a combination thereof. In certain embodiments, the subject is a Cavalier King Charles Spaniel.

### EXAMPLES

The following examples are offered by way of illustration, and not by way of limitation.

### Example 1: Canine sTGFβR2 and FGF21 Recombinant AAV Vectors

This example provides canine sTGFβR2 and FGF21 recombinant adeno-associated virus (rAAV) vectors for expression of canine sTGFβR2 and FGF21 in a cell (e.g., a canine liver cell) to which the vectors are transduced.

rAAV-sTGFβR2 comprises a rAAV genome comprising from 5′ to 3′ the following genetic elements: a 5′ ITR element, an apolipoprotein E (ApoE) binding site, a human alpha-1 antitrypsin (hAAT) promoter, a beta-globin intron sequence, a canine sTGFβR2-Fc coding sequence, a WPRE3 sequence, an SV40 polyadenylation signal, and a 3′ ITR element. The sequences of these elements are set forth in Table 1. This vector is capable of expressing a canine sTGFβR2-Fc fusion protein in a cell (e.g., a liver cell) to which the vector is transduced.

rAAV-FGF21 comprises a rAAV genome comprising from 5′ to 3′ the following genetic elements: a 5′ ITR element, an apolipoprotein E (ApoE) binding site, a human alpha-1 antitrypsin (hAAT) promoter, a beta-globin intron sequence, a canine FGF21 coding sequence, a WPRE3 sequence, an SV40 polyadenylation signal, and a 3′ ITR element. The sequences of these elements are set forth in Table 1. This vector is capable of expressing a canine FGF21 protein in a cell (e.g., a liver cell) to which the vector is transduced.

TABLE 1
Genetic Elements in rAAV-sTGFβR2 and rAAV-FGF21

| | rAAV-sTGFβR2 | rAAV-FGF21 |
|---|---|---|
| Genetic Element | SEQ ID NO: | |
| 5′ ITR element | 59 | 59 |
| ApoE binding site | 39 or 40 | 39 or 40 |
| hAAT promoter sequence | 38 | 38 |
| Beta-globin intron sequence | 48 | 48 |
| Coding sequence | 24 | 30 |
| Encoded sequence | 26 | 33 |
| WPRE3 sequence | 50 | 50 |
| SV40 polyadenylation signal | 51 | 51 |
| 3′ ITR element | 61 | 61 |
| rAAV genome (promoter to | 66 | 68 |
| polyadenylation signal) | | |
| rAAV genome | 67 | 69 |
| (5′ ITR to 3′ ITR) | | |

The rAAV vectors disclosed herein can be packaged in an AAV capsid, such as, without limitation, an AAV8 capsid. In general, viral particles were generated using standard triple transfection of HEK293T cells and either iodixanol gradient purification. CsCl purification or affinity and anion column purification. See, e.g., Davidsohn et al. (2019) Proc. Natl. Acad. Sci. 116 (47): 23505-23511; and Nass et al. (2018) Mol. Ther. Methods Clin. Dev. 9. 33-46. The packaged viral particles can be administered to a wild-type animal, or an animal suffering from a mitral valve disease.

### Example 2: sTGFβ2 and FGF21 Protein Expression in Dogs with Mitral Valve Disease

The effect of sTGFβR2 and FGF21 gene therapy was investigated in dogs with mitral valve disease. Dogs with stage B2 myxomatous mitral valve disease (MMVD) were recruited. Stage B2 MMVD refers to dogs with MMVD that have not yet developed signs of heart failure but have a moderate or loud mitral murmur due to a leaking mitral heart valve and have an enlarged heart.

Left atrial-to-aortic root ratio (LA/Ao) is the most commonly used method to evaluate left atrial (LA) size in dogs. Standard M-mode, two-dimensional Doppler echocardiographic images and video-loops were recorded with continuous ECG monitoring for all measurements. LA/Ao was measured from a right parasternal short-axis view at the heart base as previously described. The internal short-axis diameter of the aorta was measured along the commissure between the noncoronary and right coronary aortic valve cusps on the 1st frame after aortic valve closure. The internal short-axis diameter of the LA in the same frame in a line extending from and parallel to the commissure between the noncoronary and left coronary aortic valve cusps to the distant margin of the left atrium was measured. Normal LA/Ao was defined as <1.6. Stage B2 MMVD was defined as dogs having LA/Ao of 1.7-3, requiring treatment with pimobendan, but not yet in heart failure.

To examine protein expression from liver transduced with rAAV-sTGFβR2 and rAAV-FGF21 vectors, Stage B2 MMVD dogs were intravenously administered rAAV-sTGFβR2 and rAAV-FGF21, each packaged in AAV8 capsid (AAV8-sTGFβR2 and AAV8-FGF21, respectively; see, Table 1 for sequences). AAV8-sTGFβR2 was administered at a dose of 1E13, 3E13, or 5E13 vg/kg, and AAV8-FGF21 was administered at dose of 1E13 or 3E13 vg/kg. Virus was titered by ddPCR using gene specific primers for the gene of interest, (e.g., FGF21 or STGFØR2).

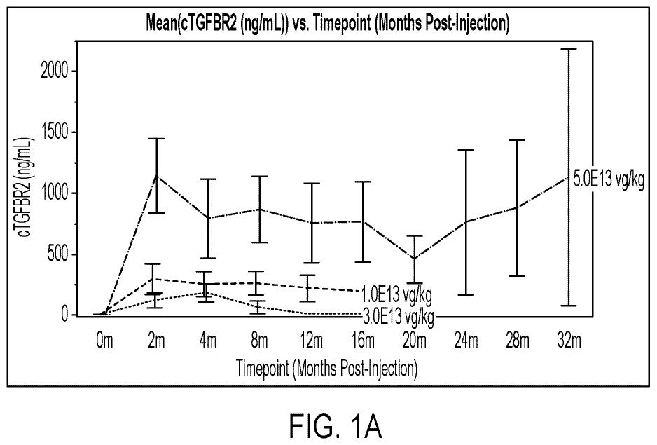

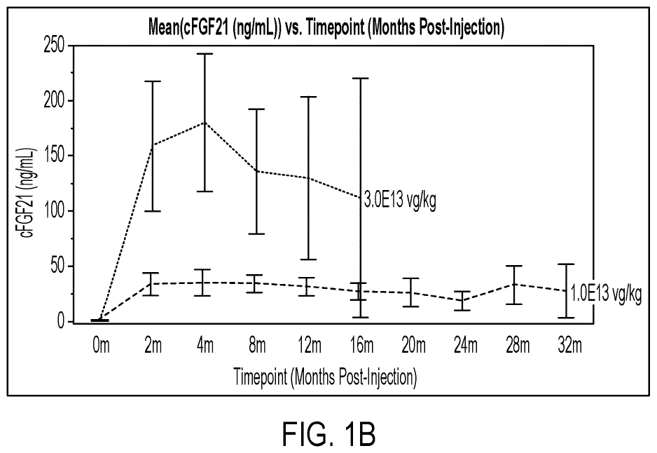

sTGFβR2-Fc and FGF21 expression was measured by ELISA using antibodies to canine TGFβR2 and FGF21, respectively. FIG. 1A shows the expression level of sTGFβR2 expression and FIG. 1B shows the expression level of FGF21 in treated dogs. As shown in FIG. 1A, dogs were administered either 1E13, 3E13, or 5E13 vg/kg of AAV8-sTGFβR2, and stable long-term expression of sTGFβR2 was achieved for over 16, 16, and 32 months, respectively. Stable long-term expression of FGF21 was also achieved for over 16 and 32 months, respectively, in dogs administered either 1E13 or 3E13 vg/kg of AAV8-FGF21 (FIG. 1B). In FIG. 1B, one dog in the 3E13 vg/kg dose cohort was a non-responder.

### Example 3: Mitral Valve Disease Gene Therapy in Dogs

Dogs with MVD typically experience an enlargement in the left atrium of their hearts due to damage caused by the malfunctioning mitral valve. LA/Ao has been shown to correlate with progression of MVD with a 0.1 increase in LA/Ao increasing the change of progression of MVD to the next stage by about 11%.

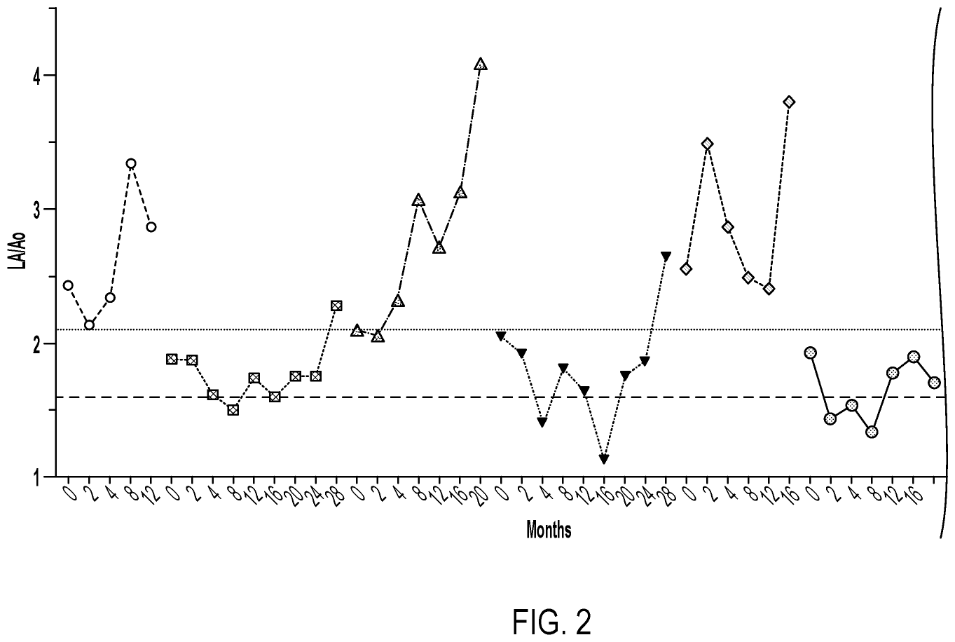

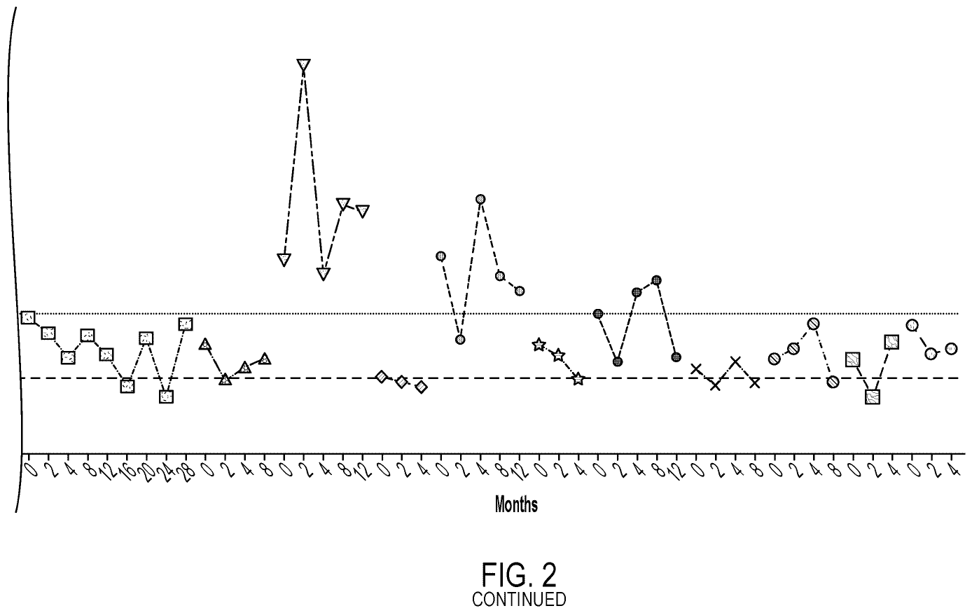

To investigate whether sTGFβR2 and FGF21 gene therapy could treat MVD in dogs, Stage B2 MMVD dogs were administered AAV8-sTGFβR2 and AAV8-FGF21 each at a dose of 1E13 vg/kg, or each at a dose of 3E13 vg/kg. LA/Ao was measured in the treated dogs over time. FIG. 2 shows the LA/Ao measurements of dogs over time, at the various indicated time points. As shown in FIG. 2, regardless of dosage, dogs with an initial LA/Ao of less than 2.1 responded to the gene therapy over time, showing a reversal of left atrium enlargement over time. In FIG. 2, the dashed line indicates entrance criteria of LA/Ao of 1.6, and the dotted line indicates LA/Ao of 2.1 under which all dogs responded to the therapy.

To date, 17 dogs have received AAV8-sTGFβR2 and AAV8-FGF21 and no adverse safety events were recorded. Four of these dogs have been in the study for over two years.

### Example 4: Combination Treatment of Mitral Valve Disease in Dogs

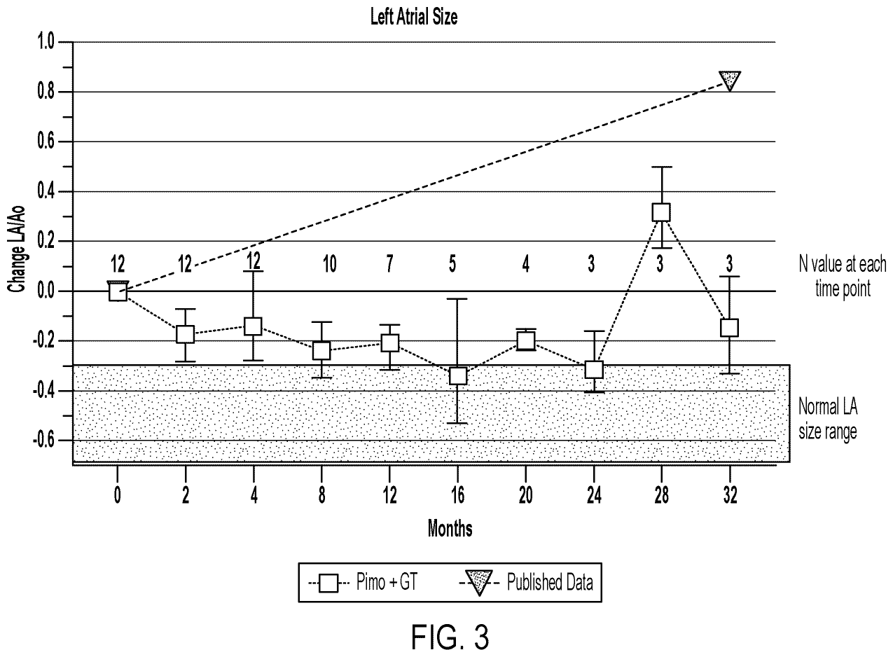

Pimobendan is the current best in class medication labeled for use in dogs to manage congestive heart failure (CHF) resulting from dilated cardiomyopathy (DCM) or degenerative MVD. Since it would be unethical to withhold standard of care in an investigational pilot study, dogs diagnosed with stage B2 MMVD and prescribed pimobendan were administered sTGFβR2 and FGF21 gene therapy. Echocardiograms were performed at baseline (0 months), 2 months post-treatment and 4 months post treatment, and every 4 months after, through 32 month-post treatment. The echocardiograms were used to measure left atrial size in reference to the aorta and fractional shortening percent (FS %) of the left ventricle. Although pimobendan has a marginal ability to reverse pathological progression of echocardiographic measurements, with published data demonstrating a 0.08 decrease in LA/Ao after being on pimobendan for 1 month (Boswood et al., J Vet Intern Med, 2018, 32 (1): 72-85), a remarkable 0.3 reversal of left atrium enlargement was observed in dogs with MVD after administration with AAV8-sTGFβR2 and AAV8-FGF21 (“GT”), which would infer a ~33% decreased chance of progression of the disease according to the same study (FIG. 3). The size of the left atrium was quantified using echocardiograms by measuring the size of the left atrium in reference to the aorta. As shown in FIG. 3, dogs treated with the combination of pimobendan together with AAV8-sTGFβR2 and AAV8-FGF21 (“Pimo +GT”) exhibited a reduction in LA/Ao of about 0.3 over 32 months. In this experiment, 12 dogs were treated at early time points, and 3 dogs were treated at final time points due to rolling admission of subjects. In contrast, based on published data (dashed line) reporting later stage LA/Ao values, use of pimobendan alone can only minimize the progressive left atrial dilation size to 0.8 over 32 months (see, e.g., Nakamura et al. J. Vet. Intern. Med. 2017, 31 (2): 316-325).

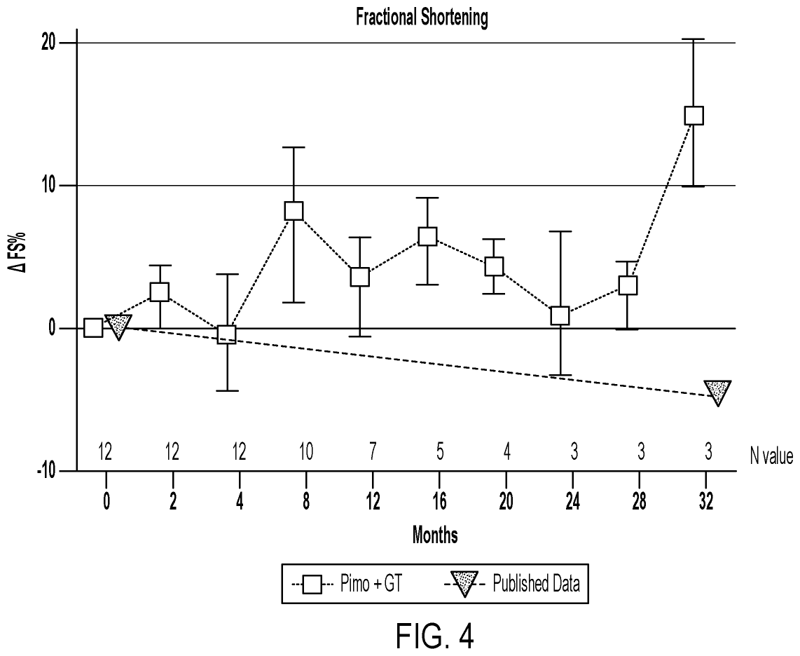

Dogs treated with the combination of pimobendan together with AAV8-sTGFβR2 and AAV8-FGF21 (“Pimo+GT”) were able to maintain cardiac function. Using fractional shortening as a measure of cardiac contractility, based on published data, treatment of dogs having MVD with pimobendan limits the decrease in contractility to about 5% over 28 months (dashed line in FIG. 4; see, e.g., Nakamura et al. J. Vet. Intern. Med. 2017, 31 (2): 316-325). As such, even under the current best in class medication, dogs with MVD typically exhibit degradation in fractional shortening. As shown in FIG. 4, dogs with MVD that received Pimo+GT exhibited an increase in fractional shortening by 3% over 28 months, representing a reversal in the progression of disease.

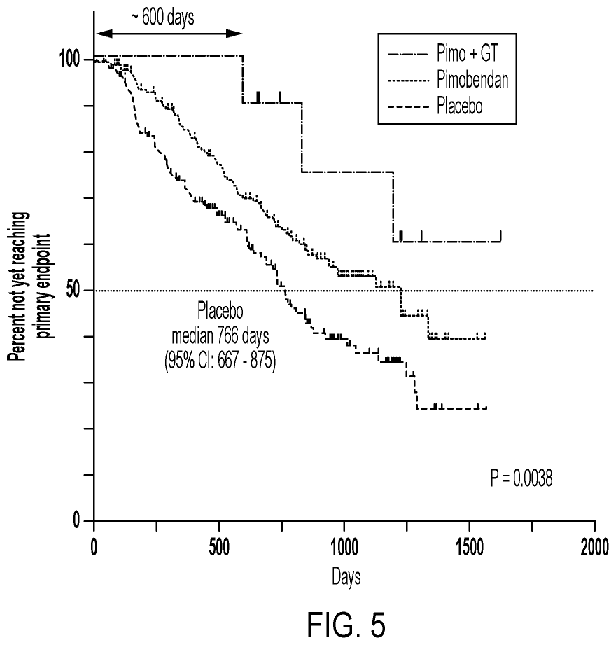

In addition, dogs with MVD that received Pimo+GT exhibited delayed progression of over 1.5 years longer compared to dogs with MVD that received standard of care. As shown in FIG. 5, dogs with MVD that received Pimo+GT exhibited an increase in time to progression of about 600 days longer compared to the published data of dogs with MVD that received pimobendan alone, and about 800 days longer compared to the published data of dogs with MVD that received placebo. Based on published data, dogs with MVD that received pimobendan alone exhibited an increase in time to progression of about 200 days over dogs with MVD that received placebo. See, e.g., Boswood et al., J Vet Intern Med, 2018, 32 (1): 72-85. Time to progression was measured based on the percentage of animals that have not yet reached the primary endpoint of onset of congestive heart failure, cardiac-related death, or euthanasia.

Importantly, no safety issues have been reported in three dogs with MVD that have been on the AAV8-sTGFβR2 and AAV8-FGF21 therapy for over three years.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entireties and for all purposes to the same extent as if each individual reference (e.g., publication or patent or patent application) was specifically and individually indicated to be incorporated by reference in its entirety for all purposes.

Other embodiments are within the following claims.

We claim:

1. A method of treating a mitral valve disease in a subject, comprising administering to the subject a gene therapy comprising a first nucleic acid encoding soluble transforming growth factor beta receptor 2 (sTGFβR2) and a second nucleic acid encoding fibroblast growth factor 21 (FGF21), wherein the subject has a left atrial to aortic root ratio (LA/Ao) of from about 1.6 to about 2.1.

2. The method of claim 1, wherein the subject has a LA/Ao of about 1.6.

3. The method of claim 1, wherein the subject has a LA/Ao of about 1.8.

4. The method of any one of claims 1-3, wherein the subject is mammal.

5. The method of any one of claims 1-4, wherein the subject is a canine.

6. The method of any one of claims 1-5, wherein the subject is a canine breed selected from the group consisting of: Cavalier King Charles Spaniel, Miniature Poodle, Shih Tzu, Maltese, Chihuahua, Cocker Spaniel, Miniature Schnauzer, Dachshund, Whippet, Pomeranian, and a combination thereof.

7. The method of any one of claims 1-6, wherein the subject is a Cavalier King Charles Spaniel.

8. The method of any one of claims 1-7, wherein the mitral valve disease comprises one or more disease or condition selected from the group consisting of: myxomatous mitral valve disease, mitral valve stenosis, mitral valve prolapse, and mitral valve regurgitation.

9. The method of any one of claims 1-8, wherein the method further comprises determining the LA/Ao of the subject prior to administration of the gene therapy, and determining the LA/Ao of the subject at a duration after administration of the gene therapy.

10. The method of any one of claims 1-9, wherein the LA/Ao of the subject after administration of the gene therapy is decreased as compared to the LA/Ao of the subject prior to administration of the gene therapy.

11. The method of any one of claims 1-10, wherein the subject has been administered an effective amount of pimobendan.

12. The method of any one of claims 1-10, further comprising administering to the subject an effective amount of pimobendan.

13. A method of treating a mitral valve disease in a subject, comprising administering to the subject a gene therapy comprising a first nucleic acid encoding soluble transforming growth factor beta receptor 2 (sTGFβR2) and a second nucleic acid encoding fibroblast growth factor 21 (FGF21); and/or an effective amount of pimobendan.

14. A method of treating a mitral valve disease in a subject that has received an effective amount of pimobendan, comprising administering to the subject a gene therapy comprising a first nucleic acid encoding soluble transforming growth factor beta receptor 2 (sTGFβR2) and a second nucleic acid encoding fibroblast growth factor 21 (FGF21).

15. The method of any one of claims 11-14, wherein the effective amount of pimobendan is 0.25 mg/kg.

16. The method of claim 12 or 13, wherein the gene therapy and pimobendan are administered simultaneously.

17. The method of any one of claims 11-16, wherein the pimobendan is administered orally, optionally wherein the effective amount of pimobendan is 0.25 mg/kg twice a day.

18. A method of determining the likelihood of successful treatment of a subject having mitral valve disease with a gene therapy comprising a first nucleic acid encoding sTGFβR2 and a second nucleic acid encoding FGF21, comprising determining the LA/Ao of a subject, wherein an LA/Ao of greater than 2.1 indicates a decreased likelihood of successful treatment with the gene therapy and an LA/Ao of from about 1.6 to about 2.1 indicates an increased likelihood of successful treatment with the gene therapy.

19. The method of claim 18, wherein successful treatment of mitral valve disease comprises a decrease in LA/Ao of the subject as compared to the LA/Ao prior to gene therapy.

20. A method of identifying a subject having mitral valve disease that is suitable for treatment with a gene therapy comprising a first nucleic acid encoding sTGFβR2 and a second nucleic acid encoding FGF21, comprising determining the LA/Ao of a subject, wherein if the subject has an LA/Ao of from about 1.6 to about 2.1, the subject is suitable for treatment with the gene therapy.

21. The method of any one of claims 18-20, wherein the subject is mammal.

22. The method of any one of claims 18-21, wherein the subject is a canine.

23. The method of claim 22, wherein the canine is a breed selected from the group consisting of: Cavalier King Charles Spaniel, Miniature Poodle, Shih Tzu, Maltese, Chihuahua, Cocker Spaniel, Miniature Schnauzer, Dachshund, Whippet, Pomeranian, and a combination thereof.

24. The method of any one of claims 18-23, wherein the subject is a Cavalier King Charles Spaniel.

25. The method of any one of claims 18-24, wherein the mitral valve disease comprises one or more disease or condition selected from the group consisting of: myxomatous mitral valve disease, mitral valve stenosis, mitral valve prolapse, and mitral valve regurgitation.

26. The method of any one of claims 1-25, wherein the gene therapy is administered intravenously.

27. The method of any one of claims 1-26, wherein the first nucleic acid comprises a first transcriptional regulatory element operably linked to the sTGFβR2 coding sequence.

28. The method of any one of claims 1-27, wherein the sTGFβR2 coding sequence comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 1, 2, 3, or 24.

29. The method any one of claims 1-28, wherein the sTGFβR2 coding sequence further comprises a heterologous or an innate secretion signal sequence, wherein the signal sequence is encoded by a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 4, 5, or 6.

30. The method of any one of claims 1-29, wherein the sTGFβR2 coding sequence comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 9, 10, 11, 12, or 25.

31. The method of any one of claims 1-27, wherein the sTGFβR2 coding sequence encodes an amino acid sequence having at least 85%, 86%, 87%, 88%, 89%, 90%, 91%, 92%, 93%, 94%, 95%, 96%, 97%, 98%, 99%, or 100% sequence identity to SEQ ID NO: 13, 14, 15, or 26.

32. The method of claim 31, wherein the sTGFβR2 coding sequence further encodes a secretion signal sequence having at least 85%, 86%, 87%, 88%, 89%, 90%, 91%, 92%, 93%, 94%, 95%, 96%, 97%, 98%, 99%, or 100% sequence identity to SEQ ID NO: 7 or 8.

33. The method of claim 31 or 32, wherein the sTGFβR2 coding sequence encodes an amino acid sequence having at least 85%, 86%, 87%, 88%, 89%, 90%, 91%, 92%, 93%, 94%, 95%, 96%, 97%, 98%, 99%, or 100% sequence identity to SEQ ID NO: 16, 17, 18, 19, or 27.

34. The method of any one of claims 1-33 wherein the second nucleic acid comprises a second transcriptional regulatory element operably linked to the FGF21 coding sequence.

35. The method of any one of claims 1-34, wherein the FGF21 coding sequence comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 28, 29, 30, or 31.

36. The method any one of claims 1-35, wherein the FGF21 coding sequence encodes an amino acid sequence having at least 85%, 86%, 87%, 88%, 89%, 90%, 91%, 92%, 93%, 94%, 95%, 96%, 97%, 98%, 99%, or 100% sequence identity to SEQ ID NO: 32, 33, or 34.

37. The method of any one of claims 34-36, wherein the first and second transcriptional regulatory element each comprises one or more ApoE binding sites and/or an hAAT promoter.

38. The method of any one of claims 34-37, wherein the first and second transcriptional regulatory element each comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 38, 39, 40, and/or 48.

39. The method of any one of claims 1-38, wherein the first and second nucleic acid each further comprises a post-transcriptional regulatory element.

40. The method of claim 39, wherein the post-transcriptional regulatory element comprises a polyadenylation signal and/or WPRE sequence.

41. The method of claim 40, wherein the polyadenylation signal is an SV40 polyadenylation signal.

42. The method of claim 40, wherein the WPRE sequence is a WPRE3 sequence.

43. The method of any one of claims 39-42, wherein the post-transcriptional regulatory element comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 50, 51, or 70.

44. The method of any one of claims 1-43, wherein the first nucleic acid is comprised within a first vector, and the second nucleic acid is comprised within a second vector.

45. The method of claim 44, wherein the first vector and/or the second vector is each a viral vector, optionally wherein each is independently selected from the group consisting of adeno-associated virus (AAV), adenovirus, retrovirus, orthomyxovirus, paramyxovirus, papovavirus, picornavirus, lentivirus, herpes simplex virus, vaccinia virus, pox virus, and alphavirus.

46. The method of claim 44 or 45, wherein:
the first vector is an AAV vector comprised within a first recombinant AAV (rAAV), wherein the first rAAV comprises an AAV capsid comprising an AAV capsid protein; and a first rAAV genome; and/or
the second vector is an AAV vector comprised within a second rAAV, wherein the second rAAV comprises an AAV capsid comprising an AAV capsid protein; and a second rAAV genome.

47. The method of any one of claims 1-46, wherein the gene therapy comprises:
a first recombinant AAV (rAAV) comprising:
an AAV capsid comprising an AAV capsid protein; and
a first rAAV genome comprising a nucleic acid encoding the amino acid sequence set forth in SEQ ID NO: 26; and
a second rAAV comprising:
an AAV capsid comprising an AAV capsid protein; and
a second rAAV genome comprising a nucleic acid encoding the amino acid sequence set forth in SEQ ID NO: 33.

48. The method of claim 46 or 47, wherein the first rAAV genome comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 66.

49. The method of any one of claims 46-48, wherein the first rAAV genome further comprises a 5′ inverted terminal repeat (5′ ITR) nucleotide sequence, and a 3′ inverted terminal repeat (3′ ITR) nucleotide sequence.

50. The method of any one of claims 46-49, wherein the 5′ ITR nucleotide sequence has at least 85%, 86%, 87%, 88%, 89%, 90%, 91%, 92%, 93%, 94%, 95%, 96%, 97%, 98%, 99%, or 100% sequence identity to SEQ ID NO: 58 or 59, and/or the 3′ ITR nucleotide sequence has at least 85%, 86%, 87%, 88%, 89%, 90%, 91%, 92%, 93%, 94%, 95%, 96%, 97%, 98%, 99%, or 100% sequence identity to SEQ ID NO: 60 or 61.

51. The method of any one of claims 46-50, wherein the first rAAV genome comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 67.

52. The method of any one of claims 46-51, wherein the second rAAV genome comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 68.

53. The method of any one of claims 46-52, wherein the second rAAV genome further comprises a 5′ inverted terminal repeat (5′ ITR) nucleotide sequence, and a 3′ inverted terminal repeat (3′ ITR) nucleotide sequence.

54. The method of claim 53, wherein the 5′ ITR nucleotide sequence has at least 85%, 86%, 87%, 88%, 89%, 90%, 91%, 92%, 93%, 94%, 95%, 96%, 97%, 98%, 99%, or 100% sequence identity to SEQ ID NO: 58 or 59, and/or the 3′ ITR nucleotide sequence has at least 85%, 86%, 87%, 88%, 89%, 90%, 91%, 92%, 93%, 94%, 95%, 96%, 97%, 98%, 99%, or 100% sequence identity to SEQ ID NO: 60 or 61.

55. The method of any one of claims 46-54, wherein the second rAAV genome comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 69.

56. The method of any one of claims 46-55, wherein the AAV capsid protein is derived from a clade A, clade B, clade C, clade D, clade E, clade F, clade G, clade H, clade I, AAVgo.1, AAV3, AAV4, AAV10, AAV11, AAV12, rh.32, rh32.33, rh.33, rh.34, BAAV, or AAV5 capsid protein, or an engineered variant thereof.

57. The method of any one of claims 46-56, wherein the AAV capsid protein comprises an amino acid sequence having at least 85%, 86%, 87%, 88%, 89%, 90%, 91%, 92%, 93%, 94%, 95%, 96%, 97%, 98%, 99%, or 100% sequence identity to SEQ ID NO: 63, 64, and/or 65.

58. The method of any one of claims 1-43, wherein the first and the second nucleic acid are comprised within a vector, optionally wherein the first and the second nucleic acid are separated by a polycistronic element.

59. The method of claim 58, wherein the vector comprises:
the first nucleic acid comprising a nucleic acid encoding the amino acid sequence set forth in SEQ ID NO: 26; and
the second nucleic acid comprising a nucleic acid encoding the amino acid sequence set forth in SEQ ID NO: 33.

60. The method of claim 58 or 59, wherein the first and the second nucleic acid are separated by a polycistronic element.

61. The method of claim 60, wherein the polycistronic element is an IRES or 2A sequence.

62. The method of claim 60 or 61, wherein the polycistronic element comprises a nucleotide sequence having at least 85%, 86%, 87%, 88%, 89%, 90%, 91%, 92%, 93%, 94%, 95%, 96%, 97%, 98%, 99%, or 100% sequence identity to SEQ ID NO: 55, 56, or 57.

63. The method of any one of claims 58-62, wherein the vector is an AAV vector comprised within a recombinant AAV (rAAV), wherein the rAAV comprises an AAV capsid comprising an AAV capsid protein; and a rAAV genome.

64. The method of claim 63, wherein the AAV capsid protein is derived from a clade A, clade B, clade C, clade D, clade E, clade F, clade G, clade H, clade I, AAVgo.1, AAV3, AAV4, AAV10, AAV11, AAV12, rh.32, rh32.33, rh.33, rh.34, BAAV, or AAV5 capsid protein, or an engineered variant thereof.

65. The method of claim 63 or 64, wherein the AAV capsid protein comprises an amino acid sequence having at least 85%, 86%, 87%, 88%, 89%, 90%, 91%, 92%, 93%, 94%, 95%, 96%, 97%, 98%, 99%, or 100% sequence identity to SEQ ID NO: 63, 64, and/or 65.


SEQUENCE LISTING (70 sequences)

SEQ ID NO: 1 - DNA, 483 bp, synthetic construct
  source 1..483: mol_type = other DNA
atcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgac

SEQ ID NO: 2 - DNA, 402 bp, synthetic construct
  source 1..402: mol_type = other DNA
aataatgacatgatggtcactgacagcaatggtgtcatcaaatttccacaattgtgtaaattttgtgatgtgagatcttccacctgtgacaaccagaaatcttgcatgagcaactgcagcattacatccatctgtgagaagccacatgaagtctgtctggctgtctggagaaagaatgatgagaacataacactagagactctctgccatgaccccaaggatacctaccatggaattgttctcgaagatgctgcctcttcgaagtgcattatgaaagaaaagaaggtgctgggggagactttctttatgtgttcctgtagctccgacgagtgcaacgactacatcatcttctctgaagaatatgccaccaacaaccctgacttgttgttagtcatattccaa

SEQ ID NO: 3 - DNA, 483 bp, synthetic construct
  source 1..483: mol_type = other DNA
atcccgccgcacgttcccaagtcggatgtggaaatggaagcccagaaagatgcatccatccacctaagctgtaataggaccatccatccactgaaacattttaacagtgatgtcatggccagcgacaatggcggtgcggtcaagcttccacagctgtgcaagttttgcgatgtgagactgtccacttgcgacaaccagaagtcctgcatgagcaactgcagcatcacggccatctgtgagaagccgcatgaagtctgcgtggccgtgtggaggaagaacgacaagaacattactctggagacggtttgccacgaccccaagctcacctaccacggcttcactctggaagatgccgcttctcccaagtgtgtcatgaaggaaaagaaaagggcgggcgagactttcttcatgtgtgcctgtaacatggaagagtgcaacgattacatcatcttttcggaagaatacaccaccagcagtcccgac

SEQ ID NO: 4 - DNA, 69 bp, synthetic construct
  source 1..69: mol_type = other DNA
atgggtcgggggctgctcaggggcctgtggccgctgcacatcgtcctgtggacgcgtatcgccagcacg

SEQ ID NO: 5 - DNA, 96 bp, synthetic construct
  source 1..96: mol_type = other DNA
atgcacagtcaagggcggggttgcaacaacacaaaacaaaacaaaacttccggacttcgacctgcagctgagaagaacatctcgcaaagcggcgtt

SEQ ID NO: 6 - DNA, 69 bp, synthetic construct
  source 1..69: mol_type = other DNA
atgggtcgggggctgctccggggcctgtggccgctgcatatcgtcctgtggacgcgcatcgccagcacg

SEQ ID NO: 7 - protein, 23 aa, synthetic construct
MGRGLLRGLWPLHIVLWTRIAST

SEQ ID NO: 8 - protein, 32 aa, synthetic construct
MHSQGRGCNNTKQNKTSGLRPAAEKNISQSGV

SEQ ID NO: 9 - DNA, 552 bp, synthetic construct
  source 1..552: mol_type = other DNA
atgggtcgggggctgctcaggggcctgtggccgctgcacatcgtcctgtggacgcgtatcgccagcacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgac

SEQ ID NO: 10 - DNA, 498 bp, synthetic construct
  source 1..498: mol_type = other DNA
atgcacagtcaagggcggggttgcaacaacacaaaacaaaacaaaacttccggacttcgacctgcagctgagaagaacatctcgcaaagcggcgttaataatgacatgatggtcactgacagcaatggtgtcatcaaatttccacaattgtgtaaattttgtgatgtgagatcttccacctgtgacaaccagaaatcttgcatgagcaactgcagcattacatccatctgtgagaagccacatgaagtctgtctggctgtctggagaaagaatgatgagaacataacactagagactctctgccatgaccccaaggatacctaccatggaattgttctcgaagatgctgcctcttcgaagtgcattatgaaagaaaagaaggtgctgggggagactttctttatgtgttcctgtagctccgacgagtgcaacgactacatcatcttctctgaagaatatgccaccaacaaccctgacttgttgttagtcatattccaa

SEQ ID NO: 11 - DNA, 471 bp, synthetic construct
  source 1..471: mol_type = other DNA
atgggtcgggggctgctccggggcctgtggccgctgcatatcgtcctgtggacgcgcatcgccagcacgaataatgacatgatggtcactgacagcaatggtgtcatcaaatttccacaattgtgtaaattttgtgatgtgagatcttccacctgtgacaaccagaaatcttgcatgagcaactgcagcattacatccatctgtgagaagccacatgaagtctgtctggctgtctggagaaagaatgatgagaacataacactagagactctctgccatgaccccaaggatacctaccatggaattgttctcgaagatgctgcctcttcgaagtgcattatgaaagaaaagaaggtgctgggggagactttctttatgtgttcctgtagctccgacgagtgcaacgactacatcatcttctctgaagaatatgccaccaacaaccctgacttgttgttagtcatattccaa

SEQ ID NO: 12 - DNA, 552 bp, synthetic construct
  source 1..552: mol_type = other DNA
atgggtcgggggctgctccggggcctgtggccgctgcatatcgtcctgtggacgcgcatcgccagcacgatcccgccgcacgttcccaagtcggatgtggaaatggaagcccagaaagatgcatccatccacctaagctgtaataggaccatccatccactgaaacattttaacagtgatgtcatggccagcgacaatggcggtgcggtcaagcttccacagctgtgcaagttttgcgatgtgagactgtccacttgcgacaaccagaagtcctgcatgagcaactgcagcatcacggccatctgtgagaagccgcatgaagtctgcgtggccgtgtggaggaagaacgacaagaacattactctggagacggtttgccacgaccccaagctcacctaccacggcttcactctggaagatgccgcttctcccaagtgtgtcatgaaggaaaagaaaagggcgggcgagactttcttcatgtgtgcctgtaacatggaagagtgcaacgattacatcatcttttcggaagaatacaccaccagcagtcccgac

SEQ ID NO: 13 - protein, 161 aa, synthetic construct
IPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 14 - protein, 161 aa, synthetic construct
IPPHVPKSDVEMEAQKDASIHLSCNRTIHPLKHFNSDVMASDNGGAVKLPQLCKFCDVRLSTCDNQKSCMSNCSITAICEKPHEVCVAVWRKNDKNITLETVCHDPKLTYHGFTLEDAASPKCVMKEKKRAGETFFMCACNMEECNDYIIFSEEYTTSSPD

SEQ ID NO: 15 - protein, 127 aa, synthetic construct
NNDMMVTDSNGVIKFPQLCKFCDVRSSTCDNQKSCMSNCSITSICEKPHEVCLAVWRKNDENITLETLCHDPKDTYHGIVLEDAASSKCIMKEKKVLGETFFMCSCSSDECNDYIIFSEEYATNNPD

SEQ ID NO: 16 - protein, 184 aa, synthetic construct
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 17 - protein, 159 aa, synthetic construct
MHSQGRGCNNTKQNKTSGLRPAAEKNISQSGVNNDMMVTDSNGVIKFPQLCKFCDVRSSTCDNQKSCMSNCSITSICEKPHEVCLAVWRKNDENITLETLCHDPKDTYHGIVLEDAASSKCIMKEKKVLGETFFMCSCSSDECNDYIIFSEEYATNNPD

SEQ ID NO: 18 - protein, 184 aa, synthetic construct
MGRGLLRGLWPLHIVLWTRIASTIPPHVPKSDVEMEAQKDASIHLSCNRTIHPLKHFNSDVMASDNGGAVKLPQLCKFCDVRLSTCDNQKSCMSNCSITAICEKPHEVCVAVWRKNDKNITLETVCHDPKLTYHGFTLEDAASPKCVMKEKKRAGETFFMCACNMEECNDYIIFSEEYTTSSPD

SEQ ID NO: 19 - protein, 150 aa, synthetic construct
MGRGLLRGLWPLHIVLWTRIASTNNDMMVTDSNGVIKFPQLCKFCDVRSSTCDNQKSCMSNCSITSICEKPHEVCLAVWRKNDENITLETLCHDPKDTYHGIVLEDAASSKCIMKEKKVLGETFFMCSCSSDECNDYIIFSEEYATNNPD

SEQ ID NO: 20 - DNA, 711 bp, synthetic construct
  source 1..711: mol_type = other DNA
cccaaaagagaaaatggaagagttcctcgcccacctgattgtcccaaatgcccagcccctgaaatgctgggagggccttcggtcttcatctttcccccgaaacccaaggacaccctcttgattgcccgaacacctgaggtcacatgtgtggtggtggatctggacccagaagaccctgaggtgcagatcagctggttcgtggacggtaagcagatgcaaacagccaagactcagcctcgtgaggagcagttcaatggcacctaccgtgtggtcagtgtcctccccattgggcaccaggactggctcaaggggaagcagttcacgtgcaaagtcaacaacaaagccctcccatccccgatcgagaggaccatctccaaggccagagggcaagcccatcagcccagtgtgtatgtcctgccgccatcccgggaggagttgagcaagaacacagtcagcttgacatgcctgatcaaagacttcttcccacctgacattgatgtggagtggcagagcaatggacagcaggagcctgagagcaagtaccgcacgaccccgccccagctggacgaggacgggtcctacttcctgtacagcaagctctctgtggacaagagccgctggcagcggggagacaccttcatatgtgcggtgatgcatgaagctctacacaaccactacacacaggaatccctctcccattctccgggtaaa

SEQ ID NO: 21 - DNA, 699 bp, synthetic construct
  source 1..699: mol_type = other DNA
cccagagggcccacaatcaagccctgtcctccatgcaaatgcccagcacctaacctcgagggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggcgttcgcatgcgcggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 22 - protein, 237 aa, synthetic construct
PKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 23 - protein, 232 aa, synthetic construct
PRGPTIKPCPPCKCPAPNLEGGPSVFIFPPKIKDVLMISLSPIVTCVVVDVSEDDPDVQISWFVNNVEVHTAQTQTHREDYNSTLRVVSALPIQHQDWMSGKAFACAVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 24 - DNA, 1113 bp, synthetic construct
  source 1..1113: mol_type = other DNA
aataatgacatgatggtcactgacagcaatggtgtcatcaaatttccacaattgtgtaaattttgtgatgtgagatcttccacctgtgacaaccagaaatcttgcatgagcaactgcagcattacatccatctgtgagaagccacatgaagtctgtctggctgtctggagaaagaatgatgagaacataacactagagactctctgccatgaccccaaggatacctaccatggaattgttctcgaagatgctgcctcttcgaagtgcattatgaaagaaaagaaggtgctgggggagactttctttatgtgttcctgtagctccgacgagtgcaacgactacatcatcttctctgaagaatatgccaccaacaaccctgacttgttgttagtcatattccaacccaaaagagaaaatggaagagttcctcgcccacctgattgtcccaaatgcccagcccctgaaatgctgggagggccttcggtcttcatctttcccccgaaacccaaggacaccctcttgattgcccgaacacctgaggtcacatgtgtggtggtggatctggacccagaagaccctgaggtgcagatcagctggttcgtggacggtaagcagatgcaaacagccaagactcagcctcgtgaggagcagttcaatggcacctaccgtgtggtcagtgtcctccccattgggcaccaggactggctcaaggggaagcagttcacgtgcaaagtcaacaacaaagccctcccatccccgatcgagaggaccatctccaaggccagagggcaagcccatcagcccagtgtgtatgtcctgccgccatcccgggaggagttgagcaagaacacagtcagcttgacatgcctgatcaaagacttcttcccacctgacattgatgtggagtggcagagcaatggacagcaggagcctgagagcaagtaccgcacgaccccgccccagctggacgaggacgggtcctacttcctgtacagcaagctctctgtggacaagagccgctggcagcggggagacaccttcatatgtgcggtgatgcatgaagctctacacaaccactacacacaggaatccctctcccattctccgggtaaa

SEQ ID NO: 25 - DNA, 1182 bp, synthetic construct
  source 1..1182: mol_type = other DNA
atgggtcgggggctgctccggggcctgtggccgctgcatatcgtcctgtggacgcgcatcgccagcacgaataatgacatgatggtcactgacagcaatggtgtcatcaaatttccacaattgtgtaaattttgtgatgtgagatcttccacctgtgacaaccagaaatcttgcatgagcaactgcagcattacatccatctgtgagaagccacatgaagtctgtctggctgtctggagaaagaatgatgagaacataacactagagactctctgccatgaccccaaggatacctaccatggaattgttctcgaagatgctgcctcttcgaagtgcattatgaaagaaaagaaggtgctgggggagactttctttatgtgttcctgtagctccgacgagtgcaacgactacatcatcttctctgaagaatatgccaccaacaaccctgacttgttgttagtcatattccaacccaaaagagaaaatggaagagttcctcgcccacctgattgtcccaaatgcccagcccctgaaatgctgggagggccttcggtcttcatctttcccccgaaacccaaggacaccctcttgattgcccgaacacctgaggtcacatgtgtggtggtggatctggacccagaagaccctgaggtgcagatcagctggttcgtggacggtaagcagatgcaaacagccaagactcagcctcgtgaggagcagttcaatggcacctaccgtgtggtcagtgtcctccccattgggcaccaggactggctcaaggggaagcagttcacgtgcaaagtcaacaacaaagccctcccatccccgatcgagaggaccatctccaaggccagagggcaagcccatcagcccagtgtgtatgtcctgccgccatcccgggaggagttgagcaagaacacagtcagcttgacatgcctgatcaaagacttcttcccacctgacattgatgtggagtggcagagcaatggacagcaggagcctgagagcaagtaccgcacgaccccgccccagctggacgaggacgggtcctacttcctgtacagcaagctctctgtggacaagagccgctggcagcggggagacaccttcatatgtgcggtgatgcatgaagctctacacaaccactacacacaggaatccctctcccattctccgggtaaa

SEQ ID NO: 26 - protein, 371 aa, synthetic construct
NNDMMVTDSNGVIKFPQLCKFCDVRSSTCDNQKSCMSNCSITSICEKPHEVCLAVWRKNDENITLETLCHDPKDTYHGIVLEDAASSKCIMKEKKVLGETFFMCSCSSDECNDYIIFSEEYATNNPDLLLVIFQPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 27 - protein, 394 aa, synthetic construct
MGRGLLRGLWPLHIVLWTRIASTNNDMMVTDSNGVIKFPQLCKFCDVRSSTCDNQKSCMSNCSITSICEKPHEVCLAVWRKNDENITLETLCHDPKDTYHGIVLEDAASSKCIMKEKKVLGETFFMCSCSSDECNDYIIFSEEYATNNPDLLLVIFQPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 28 - DNA, 627 bp, synthetic construct
  source 1..627: mol_type = other DNA
atggactcggacgagaccgggttcgagcactcaggactgtgggtttctgtgctggctggtcttctgctgggagcctgccaggcacaccccatccctgactccagtcctctcctgcaattcgggggccaagtccggcagcggtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagctgcttcttgaggacggatacaatgtttaccagtccgaagcccacggcctcccgctgcacctgccagggaacaagtccccacaccgggaccctgcaccccgaggaccagctcgcttcctgccactaccaggcctgccccccgcactcccggagccacccggaatcctggccccccagccccccgatgtgggctcctcggaccctctgagcatggtgggaccttcccagggccgaagccccagctacgcttcc

SEQ ID NO: 29 - DNA, 633 bp, synthetic construct
  source 1..633: mol_type = other DNA
atgggctgggccgaggccgggttcgagcacctgggactgtgggtccctgtgctggctgtgcttttgctggaagcctgccgggcacatccgatccctgactccagccccctcctacaatttggaggtcaagttcgacagcggtacctctacaccgacgatgcccaggagacagaggcccacctagagatcagggccgatggcacagtggtgggggctgcccgccagagccctgaaagtctcctggagctgaaagccctaaagccaggggtcattcaaatcttgggagtcaaaacatccaggttcctgtgccagggcccagatgggacactatatggctcgctccatttcgaccctgtggcctgcagtttccgagaactgcttcttgaggatgggtacaacatctaccactccgagacccttggtctcccgcttcgcctgcgcccccacaactccgcataccgggacttggcaccccgcgggcctgcccgcttcctgccactgccaggcctgcttccagcacccccagagcctccagggatcctggccccggagcctcctgacgtgggctcctcggaccctctgagcatggtggggccttcacagggccggagtcccagctatgcttcctgatag

SEQ ID NO: 30 - DNA, 627 bp, synthetic construct
  source 1..627: mol_type = other DNA
atggggtgggccgaggccgggttcgaacatcttgggctttgggtcccagttctcgctgtcctgcttctggaagcttgccgcgcacatccaatccccgactcttcacccctgctgcagttcggaggacaagtgcgccagagatacctgtacactgatgacgcgcaggaaaccgaggcgcacttggaaattagggcagatgggaccgtggtcggagcagccaggcaaagtcctgagagcctgctcgaattgaaagccctcaagcccggcgtgatccaaatcctgggggttaagacatccagatttctctgccaagggccggatggaaccttgtacggtagccttcatttcgacccagtggcttgttcctttcgagagttgctcctggaagacggatacaatatctaccactccgaaactctcggattgcccttgaggctgagacctcacaactctgcttaccgagacttggctcccaggggtcctgcaagattcctgccgctgccgggattgcttcctgccccaccagagccccctggaatccttgcgccagaaccacccgatgttggctccagtgacccactttcaatggttggcccttcccaaggtagaagtccctcatacgcatcc

SEQ ID NO: 31 - DNA, 633 bp, synthetic construct
  source 1..633: mol_type = other DNA
atggaatggatgagatctagagttgggaccctgggactgtgggtccgactgctgctggctgtcttcctgctgggggtctaccaagcataccccatccctgactccagccccctcctccagtttgggggtcaagtccggcagaggtacctctacacagatgacgaccaagacactgaagcccacctggagatcagggaggatggaacagtggtaggcgcagcacaccgcagtccagaaagtctcctggagctcaaagccttgaagccaggggtcattcaaatcctgggtgtcaaagcctctaggtttctttgccaacagccagatggagctctctatggatcgcctcactttgatcctgaggcctgcagcttcagagaactgctgctggaggacggttacaatgtgtaccagtctgaagcccatggcctgcccctgcgtctgcctcagaaggactccccaaaccaggatgcaacatcctggggacctgtgcgcttcctgcccatgccaggcctgctccacgagccccaagaccaagcaggattcctgcccccagagcccccagatgtgggctcctctgaccccctgagcatggtagagcctttacagggccgaagccccagctatgcgtcctga

SEQ ID NO: 32 - protein, 209 aa, synthetic construct
MDSDETGFEHSGLWVSVLAGLLLGACQAHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 33 - protein, 209 aa, synthetic construct
MGWAEAGFEHLGLWVPVLAVLLLEACRAHPIPDSSPLLQFGGQVRQRYLYTDDAQETEAHLEIRADGTVVGAARQSPESLLELKALKPGVIQILGVKTSRFLCQGPDGTLYGSLHFDPVACSFRELLLEDGYNIYHSETLGLPLRLRPHNSAYRDLAPRGPARFLPLPGLLPAPPEPPGILAPEPPDVGSSDPLSMVGPSQGRSPSYAS

SEQ ID NO: 34 - protein, 210 aa, synthetic construct
MEWMRSRVGTLGLWVRLLLAVFLLGVYQAYPIPDSSPLLQFGGQVRQRYLYTDDDQDTEAHLEIREDGTVVGAAHRSPESLLELKALKPGVIQILGVKASRFLCQQPDGALYGSPHFDPEACSFRELLLEDGYNVYQSEAHGLPLRLPQKDSPNQDATSWGPVRFLPMPGLLHEPQDQAGFLPPEPPDVGSSDPLSMVEPLQGRSPSYAS

SEQ ID NO: 35 - DNA, 1174 bp, synthetic construct
  source 1..1174: mol_type = other DNA
ttggctccggtgcccgtcagtgggcagagcgcacatcgcccacagtccccgagaagttgtggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggt

SEQ ID NO: 36 - DNA, 231 bp, synthetic construct
  source 1..231: mol_type = other DNA
aggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacaca

SEQ ID NO: 37 - DNA, 544 bp, synthetic construct
  source 1..544: mol_type = other DNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 38 - DNA, 393 bp, synthetic construct
  source 1..393: mol_type = other DNA
atcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtg

SEQ ID NO: 39 - DNA, 154 bp, synthetic construct
  source 1..154: mol_type = other DNA
cagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagc

SEQ ID NO: 40 - DNA, 504 bp, synthetic construct
  source 1..504: mol_type = other DNA
cagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaagtgtgcgatatgcagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaaggatgcgtaatgcagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagc

SEQ ID NO: 41 - DNA, 100 bp, synthetic construct
  source 1..100: mol_type = other DNA
ccaaaatcaacgggactttccaaaatgtcgtaataaccccgccccgttgacgcaaatgggcggtaggcgtgtacggtgggaggtctatataagcagagct

SEQ ID NO: 42 - DNA, 1873 bp, synthetic construct
  source 1..1873: mol_type = other DNA
gatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaatt

SEQ ID NO: 43 - DNA, 913 bp, synthetic construct
  source 1..913: mol_type = other DNA
ggcatcctaaaaaatattcagtggaaacgtaaaaacattaaagactgattaaacatcgcagcatgacacagatttagcaactgagcataaataatttgactcggatactgctccaaaatccgaagaggaccaatttcttccaggaggacaactacctcgtcctctgcagacccctctcctcggcagctgaaggagtgtggccaatctgcctccacctccccgcggaccccctactctcaggacctcctgcagcaccccaaactggaagtggccgctgcagacccaaggacgaggggcacgcgggagccggcagccctagtggagcggttggagatgttgaggtgggagggtcacccaggtggggtgaggctggggtaggtagcggagtgaacggcttccgaagctctgggccgcccccaggttggactaagcaggcgctctgtcttcgcccccgcccagggtgggcgtctcctgaggactccccgccacacctgacccgagaccgcgcgcccagcctagaacgcttccccgacccagcgtagggccgccgcgactggcgggcgagggtcggcgggaggcctggcgaacccgggggcgggaccaggcgggcaaggcccggctgccgcagcgccgctctgcgcgaggcggctccgccgcggcggagggatacggcgcaccatatatatatcgcggggcgcagactcgcgctccggcagtggtgctgggagtgtcgtggacgccgtgccgttactcgtagtcaggcggcggcgcaggcggcggcggcggcatagcgcacagcgcgccttagcagcagcagcagcagcagcggcatcggaggtacccccgccgtcgcagcccccgcgctggtgcagccaccctcgctccctctgctcttcctcccttcgctcgcacc

SEQ ID NO: 44 - DNA, 1246 bp, synthetic construct
  source 1..1246: mol_type = other DNA
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcgg

SEQ ID NO: 45 - DNA, 1061 bp, synthetic construct
  source 1..1061: mol_type = other DNA
tagggaggtcctgcacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcgggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactaaaacaggtaagtccggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgatgcctctactaaccatgttcatgttttctttttttttctacaggtcctgggtgacgaacag

SEQ ID NO: 46 - DNA, 953 bp, synthetic construct
  source 1..953: mol_type = other DNA
aattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaag

SEQ ID NO: 47 - DNA, 82 bp, synthetic construct
  source 1..82: mol_type = other DNA
gtaagggtttaagggatggttggttggtggggtattaatgtttaattacctggagcacctgcctgaaatcactttttttcag

SEQ ID NO: 48 - DNA, 476 bp, synthetic construct
  source 1..476: mol_type = other DNA
gtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 49 - DNA, 592 bp, synthetic construct
  source 1..592: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 50 - DNA, 248 bp, synthetic construct
  source 1..248: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtt

SEQ ID NO: 51 - DNA, 132 bp, synthetic construct
  source 1..132: mol_type = other DNA
gctttatttgtgaaatttgtgatgctattgctttatttgtaaccattataagctgcaataaacaagttaacaacaacaattgcattcattttatgtttcaggttcagggggagatgtgggaggttttttaaa

SEQ ID NO: 52 - DNA, 510 bp, synthetic construct
  source 1..510: mol_type = other DNA
acgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccga

SEQ ID NO: 53 - DNA, 387 bp, synthetic construct
  source 1..387: mol_type = other DNA
tggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 54 - DNA, 479 bp, synthetic construct
  source 1..479: mol_type = other DNA
acgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 55 - DNA, 54 bp, synthetic construct
  source 1..54: mol_type = other DNA
gagggccgcggcagcctgctgacctgcggcgacgtggaggaaaaccccggcccc

SEQ ID NO: 56 - DNA, 59 bp, synthetic construct
  source 1..59: mol_type = other DNA
ggatctggcgccaccaacttctctctgctgaagcaggccggcgacgtggaggagaaccc

SEQ ID NO: 57 - DNA, 581 bp, synthetic construct
  source 1..581: mol_type = other DNA
gcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagccacc

SEQ ID NO: 58 - DNA, 130 bp, synthetic construct
  source 1..130: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 59 - DNA, 145 bp, synthetic construct
  source 1..145: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 60 - DNA, 141 bp, synthetic construct
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 61 - DNA, 145 bp, synthetic construct
  source 1..145: mol_type = other DNA
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 62 - DNA, 2214 bp, synthetic construct
  source 1..2214: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctg

SEQ ID NO: 63 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 64 - protein, 601 aa, synthetic construct
TAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 65 - protein, 535 aa, synthetic construct
MAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 66 - DNA, 3196 bp, synthetic construct
  source 1..3196: mol_type = other DNA
cagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaagtgtgcgatatgcagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaaggatgcgtaatgcagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaggcatagctctagactgtagtacgtcgacgagctccctatagtgagtcgtattagaggccgactgacccggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatctcgagtgatccagatctaagcttctctggtcaccgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaagcggccgcatcgccaccatgggtcgggggctgctccggggcctgtggccgctgcatatcgtcctgtggacgcgcatcgccagcacgaataatgacatgatggtcactgacagcaatggtgtcatcaaatttccacaattgtgtaaattttgtgatgtgagatcttccacctgtgacaaccagaaatcttgcatgagcaactgcagcattacatccatctgtgagaagccacatgaagtctgtctggctgtctggagaaagaatgatgagaacataacactagagactctctgccatgaccccaaggatacctaccatggaattgttctcgaagatgctgcctcttcgaagtgcattatgaaagaaaagaaggtgctgggggagactttctttatgtgttcctgtagctccgacgagtgcaacgactacatcatcttctctgaagaatatgccaccaacaaccctgacttgttgttagtcatattccaacccaaaagagaaaatggaagagttcctcgcccacctgattgtcccaaatgcccagcccctgaaatgctgggagggccttcggtcttcatctttcccccgaaacccaaggacaccctcttgattgcccgaacacctgaggtcacatgtgtggtggtggatctggacccagaagaccctgaggtgcagatcagctggttcgtggacggtaagcagatgcaaacagccaagactcagcctcgtgaggagcagttcaatggcacctaccgtgtggtcagtgtcctccccattgggcaccaggactggctcaaggggaagcagttcacgtgcaaagtcaacaacaaagccctcccatccccgatcgagaggaccatctccaaggccagagggcaagcccatcagcccagtgtgtatgtcctgccgccatcccgggaggagttgagcaagaacacagtcagcttgacatgcctgatcaaagacttcttcccacctgacattgatgtggagtggcagagcaatggacagcaggagcctgagagcaagtaccgcacgaccccgccccagctggacgaggacgggtcctacttcctgtacagcaagctctctgtggacaagagccgctggcagcggggagacaccttcatatgtgcggtgatgcatgaagctctacacaaccactacacacaggaatccctctcccattctccgggtaaataataggctagcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaa

SEQ ID NO: 67 - DNA, 4611 bp, synthetic construct
  source 1..4611: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctatgaattctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaagtgtgcgatatgcagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaaggatgcgtaatgcagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaggcatagctctagactgtagtacgtcgacgagctccctatagtgagtcgtattagaggccgactgacccggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatctcgagtgatccagatctaagcttctctggtcaccgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaagcggccgcatcgccaccatgggtcgggggctgctccggggcctgtggccgctgcatatcgtcctgtggacgcgcatcgccagcacgaataatgacatgatggtcactgacagcaatggtgtcatcaaatttccacaattgtgtaaattttgtgatgtgagatcttccacctgtgacaaccagaaatcttgcatgagcaactgcagcattacatccatctgtgagaagccacatgaagtctgtctggctgtctggagaaagaatgatgagaacataacactagagactctctgccatgaccccaaggatacctaccatggaattgttctcgaagatgctgcctcttcgaagtgcattatgaaagaaaagaaggtgctgggggagactttctttatgtgttcctgtagctccgacgagtgcaacgactacatcatcttctctgaagaatatgccaccaacaaccctgacttgttgttagtcatattccaacccaaaagagaaaatggaagagttcctcgcccacctgattgtcccaaatgcccagcccctgaaatgctgggagggccttcggtcttcatctttcccccgaaacccaaggacaccctcttgattgcccgaacacctgaggtcacatgtgtggtggtggatctggacccagaagaccctgaggtgcagatcagctggttcgtggacggtaagcagatgcaaacagccaagactcagcctcgtgaggagcagttcaatggcacctaccgtgtggtcagtgtcctccccattgggcaccaggactggctcaaggggaagcagttcacgtgcaaagtcaacaacaaagccctcccatccccgatcgagaggaccatctccaaggccagagggcaagcccatcagcccagtgtgtatgtcctgccgccatcccgggaggagttgagcaagaacacagtcagcttgacatgcctgatcaaagacttcttcccacctgacattgatgtggagtggcagagcaatggacagcaggagcctgagagcaagtaccgcacgaccccgccccagctggacgaggacgggtcctacttcctgtacagcaagctctctgtggacaagagccgctggcagcggggagacaccttcatatgtgcggtgatgcatgaagctctacacaaccactacacacaggaatccctctcccattctccgggtaaataataggctagcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaagccacgcgtgaaaagggggggttggatttcgcttgttgcataggttggtctcaaactcctggcctcaagtgattctcctgcctctgcctcccaaagtgctgagattacaggtgtgaggcaccatgccaggtctcttactgtttgtaattaaatacatacacattttgtgtgtttgtgtgcacctttataaagtcaaaggtgatagtaacccatttaagttcctactcaattttactttccagggataactaactactttttctttttgagatggagtctcgctgtgtagcccaggctggagtgcagtggcaccatctcggctcactgcaagctcctcctccctggttcacgctattctcctgcctcagcctccccaacaactaggactacaggctcacctcgccatacctggctaattttttgtatttttagtagagacagggtttcactgtgttagccaggatggtctcgatctcctgaccttgtgatccgcctgcctctgcctcccaaagtgctgggattacaggcatgagcaacctcacccagctgggataactactttttacaggttgatattcttttggacttttcccctgtgtaaaaatatactatatttgttatgtacatattatgtacatacagacacaaattggaccattctcagtataatgattctcaggttttttttttttttttgaggtggggaactagataattatggacatctttccatactagcatatcaatatctacctcattctttttaatatttttgctagtattccattgtatgaatgtcctatgatttacttaacctgtccatcaatatttgtttccaggtttttgctattataatgctgctgcaaagtacatcctcacacatctttattttgtctattcatatttctgtaagataggttactaaagttggaactgccaaattaacactatcatactattttgttttttaattttaattttttaaaaaatgtaaaatgtgcaatttcaagaggagaaacttgaacacaaggagcaaaatctatttttataacatcctattaaaagcttgctttacataaagattttgaaagaatagcataaatacatgactagtcaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 68 - DNA, 2640 bp, synthetic construct
  source 1..2640: mol_type = other DNA
cagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaagtgtgcgatatgcagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaaggatgcgtaatgcagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaggcatagctctagactgtagtacgtcgacgagctccctatagtgagtcgtattagaggccgactgacccggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatctcgaggatccagatctaagcttctctggtcaccgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaagcggccgcatcgccaccatggggtgggccgaggccgggttcgaacatcttgggctttgggtcccagttctcgctgtcctgcttctggaagcttgccgcgcacatccaatccccgactcttcacccctgctgcagttcggaggacaagtgcgccagagatacctgtacactgatgacgcgcaggaaaccgaggcgcacttggaaattagggcagatgggaccgtggtcggagcagccaggcaaagtcctgagagcctgctcgaattgaaagccctcaagcccggcgtgatccaaatcctgggggttaagacatccagatttctctgccaagggccggatggaaccttgtacggtagccttcatttcgacccagtggcttgttcctttcgagagttgctcctggaagacggatacaatatctaccactccgaaactctcggattgcccttgaggctgagacctcacaactctgcttaccgagacttggctcccaggggtcctgcaagattcctgccgctgccgggattgcttcctgccccaccagagccccctggaatccttgcgccagaaccacccgatgttggctccagtgacccactttcaatggttggcccttcccaaggtagaagtccctcatacgcatcctaataggctagcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaa

SEQ ID NO: 69 - DNA, 4596 bp, synthetic construct
  source 1..4596: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctaccggtgaaaagggggggttggatttcgcttgttgcataggttggtctcaaactcctggcctcaagtgattctcctgcctctgcctcccaaagtgctgagattacaggtgtgaggcaccatgccaggtctcttactgtttgtaattaaatacatacacattttgtgtgtttgtgtgcacctttataaagtcaaaggtgatagtaacccatttaagttcctactcaattttactttccagggataactaactactttttctttttgagatggagtctcgctgtgtagcccaggctggagtgcagtggcaccatctcggctcactgcaagctcctcctccctggttcacgctattctcctgcctcagcctccccaacaactaggactacaggctcacctcgccatacctggctaattttttgtatttttagtagagacagggtttcactgtgttagccaggatggtctcgatctcctgaccttgtgatccgcctgcctctgcctcccaaagtgctgggattacaggcatgagcaacctcacccagctgggataactactttttacaggttgatattcttttggacttttcccctgtgtaaaaatatactatatttgttatgtacatattatgtacatacagacacaaattggaccattctcagtataatgattctcaggttttttttttttttttgaggtggggaactagataattatggacatctttccatactagcatatcaatatctacctcattctttttaatatttttgctagtgttactgtccaatggaattctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaagtgtgcgatatgcagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaaggatgcgtaatgcagtctacagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagcaggcatagctctagactgtagtacgtcgacgagctccctatagtgagtcgtattagaggccgactgacccggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatctcgaggatccagatctaagcttctctggtcaccgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaagcggccgcatcgccaccatggggtgggccgaggccgggttcgaacatcttgggctttgggtcccagttctcgctgtcctgcttctggaagcttgccgcgcacatccaatccccgactcttcacccctgctgcagttcggaggacaagtgcgccagagatacctgtacactgatgacgcgcaggaaaccgaggcgcacttggaaattagggcagatgggaccgtggtcggagcagccaggcaaagtcctgagagcctgctcgaattgaaagccctcaagcccggcgtgatccaaatcctgggggttaagacatccagatttctctgccaagggccggatggaaccttgtacggtagccttcatttcgacccagtggcttgttcctttcgagagttgctcctggaagacggatacaatatctaccactccgaaactctcggattgcccttgaggctgagacctcacaactctgcttaccgagacttggctcccaggggtcctgcaagattcctgccgctgccgggattgcttcctgccccaccagagccccctggaatccttgcgccagaaccacccgatgttggctccagtgacccactttcaatggttggcccttcccaaggtagaagtccctcatacgcatcctaataggctagcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaagccacgcgtgtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaagtgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagcactagtaaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 70 - DNA, 235 bp, synthetic construct
  source 1..235: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgaca